(19) United States
(12) Patent Application Publication
(10) Pub. No.: US 2021/0317385 A1
(43) Pub. Date: Oct. 14, 2021

(54) CONTACT LENS SOLUTIONS AND KITS

(71) Applicant: Aldeyra Therapeutics, Inc., Lexington, MA (US)

(72) Inventors: Susan MACDONALD, Danvers, MA (US); Stephen Gitu MACHATHA, Wilmington, MA (US)

(21) Appl. No.: 17/282,301

(22) PCT Filed: Oct. 2, 2019
(86) PCT No.: PCT/US2019/054263

§ 371 Date: Apr. 1, 2021

Related U.S. Application Data
(60) Provisional application No. 62/740,304, filed on Oct. 2, 2018.

(51) Int. Cl. C11D 3/00; C11D 7/26; A61K 9/00; A61K 47/40; A61K 47/02; C11D 3/12

(52) U.S. Cl. CPC C11D 3/0078; C11D 7/268; A61K 9/0048; C11D 3/1226; A61K 47/02; C11D 3/0047; A61K 47/40

(57) ABSTRACT

The present disclosure relates to contact lens solution comprising cyclodextrin, ophthalmic solutions comprising cy-clodextrin for treating disorders or conditions associated with wearing of contact lenses, methods of treating such disorders or conditions using the ophthalmic cyclodextrin solutions, and kits and combination products thereof.

### 1. CROSS-REFERENCE TO RELATED APPLICATIONS

This application claims the benefit under 35 U.S.C. § 119(e) of U.S. Provisional Application No. 62/740,304, filed Oct. 2, 2018, the contents of all of which are incorporated herein in their entireties by reference thereto.

### 2. BACKGROUND

Contact lenses are used to treat common refractive errors resulting in vision problems, such as myopia, hyperopia, astigmatism and presbyopia. Contact lenses are also prescribed for vision problems difficult to correct with regular glasses, including refractive errors due to aphakia, keratoconus, irregular cornea, and high anisometropia. However, wearing of contact lenses can also lead to complications, including corneal neovascularization, contact lens-induced peripheral ulcer (CLPU), microbial keratitis, Giant Papillary conjunctivitis, endothelial polymegethism, meibomitis, pingueculitis, ptosis, deep stromal corneal opacities, corneal hypoxia, epithelial microcoysts, excess mucus production, dry eye syndrome, corneal edema, non-microbial-associated red eye (contact lens acute red eye), and allergy aggravation. In addition, the contact lens wearer may suffer from contact lens associated discomfort (CLD), including irritation from corneal abrasion and contact lens associated allergy.

Treatments for some of these complications are straightforward, such as discontinuing use of contact lens or switching to different contact lens material. In some instances, contact lens related complications require use of pharmaceutically active agents, such an antibiotic or anti-fungal agents for treating any underlying microbial infections, anti-histamines, steroids, non-steroidal anti-inflammatory drugs (NSAIDS), and immune modulators (e.g., cyclosporine). Many of the pharmaceutically acting agents are available as topical ophthalmic formulations. However, administering these agents can have undesirable side effects. For example, ophthalmic steroids can lead to elevated intraocular pressure, increased risk of cataract formation, enhanced sensitivity to light, and systemic effects following chronic use. Side effects of immune modulator cyclosporine administered ophthalmically include ocular (e.g., conjunctival) hyperemia, ocular irritation, and eyelid erythema. Accordingly, desirable are alternative therapies for prevention and/or treatment of disorders or conditions associated with wearing of contact lenses.

### 3. SUMMARY

The present disclosure relates to a contact lens solution comprising a cyclodextrin, an ophthalmic solution having cyclodextrin as a pharmaceutically active agent, and uses of the ophthalmic solution to prevent, treat, or reduce the risk of disorders or conditions associated with wearing of contact lenses. In one aspect, the contact lens solution and/or ophthalmic solution is provided as a combination, for example a combination product, package or kit.

In some embodiments, a contact lens solution comprises cyclodextrin, wherein the contact lens solution is substantially free of a disinfecting agent, antiseptic agent and/or a preservative. In some embodiments, the contact lens solution comprises cyclodextrin, wherein the contact lens solution contains a disinfecting agent, antiseptic agent and/or a preservative.

In some embodiments, a combination, for example a combination product, package or kit, comprises a first contact lens solution comprising cyclodextrin, wherein the first contact lens solution contains a disinfecting agent, antiseptic agent and/or a preservative, and a second contact lens solution, wherein the second contact lens solution is substantially free of a disinfecting agent, antiseptic agent and/or a preservative.

In some embodiments, the combination, for example a combination product, a contact lens package or contact lens kit, comprises: (a) a first contact lens solution comprising cyclodextrin, wherein the first contact lens solution contains a disinfecting agent, antiseptic agent and/or a preservative; (b) a contact lens, wherein the contact lens is in contact with the first contact lens solution; and (c) a second contact lens solution, wherein the second contact lens solution is substantially free of a disinfecting agent, antiseptic agent and/or a preservative. In some embodiments, the second contact lens solution is provided in a container, disperser or vial separate from the first contact lens solution.

In some embodiments, a combination, for example a combination product, package, or kit, comprises: (a) a contact lens solution comprising cyclodextrin; and (b) an ophthalmic solution comprising cyclodextrin. In some embodiments, the contact lens solution contains a disinfecting agent, antiseptic agent, and/or a preservative. In some embodiments, the contact lens solution is substantially free of a disinfecting agent, antiseptic agent, and/or a preservative. In some embodiments, the ophthalmic solution has cyclodextrin as the only or sole pharmaceutically active agent.

In some embodiments, a combination, for example a combination product, a contact lens package or a contact lens kit, comprises: (a) a contact lens solution comprising cyclodextrin; (b) a contact lens, wherein the contact lens is in contact with the contact lens solution; and (c) an ophthalmic solution comprising cyclodextrin. In some embodiments, the contact lens solution contains a disinfecting agent, antiseptic agent, and/or a preservative. In some embodiments, the contact lens solution is substantially free of a disinfecting agent, antiseptic agent, and/or a preservative. In some embodiments, the ophthalmic solution has cyclodextrin as the only or sole pharmaceutically active agent.

In some embodiments, the contact lens solution has cyclodextrin at a concentration sufficient to provide a film or layer of cyclodextrin on a contact lens to act as a mucomimetic. In some embodiments, the concentration of cyclodextrin in the contact lens solution is sufficient to bind and reduce the concentration of and/or reduce the bioactivity of an eye allergen, an inflammatory mediator, and/or toxic aldehyde.

In some embodiments, the contact lens solution contains one or more excipients or additives selected from a tonicity agent, buffering agent, wetting agent, viscosity enhancing agent, lubricating agent, chelating agent, and antioxidant. As discussed herein, in some embodiments, the contact lens solution, where appropriate, contains a disinfecting agent, antiseptic agent, and/or a preservative.

In some embodiments, the contact lens solution contains cyclodextrin at a concentration of about 0.1% w/v to about 50% w/v, about 0.5% w/v to about 45% w/v, about 1% w/v to about 40% w/v, about 2% w/v to about 35% w/v, about 5% w/v to about 30% w/v, about 10% w/v to about 25% w/v, or about 15% to about 20% w/v.

In some embodiments, the contact lens solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

In some embodiments, the ophthalmic solution contains cyclodextrin at a concentration sufficient to provide a film or layer of cyclodextrin on the eye to act as a mucomimetic. In some embodiments, the concentration of cyclodextrin in the ophthalmic solution is sufficient to bind and reduce the concentration of and/or reduce the bioactivity of an eye allergen, an inflammatory mediator, and/or toxic aldehyde. In some embodiments, the ophthalmic solution contains cyclodextrin at a concentration which is effective to treat a disorder or condition associated with a wearing contact lens.

In some embodiments, the ophthalmic solution contains cyclodextrin at a concentration of about 0.1% w/v to about 50% w/v, about 0.5% w/v to about 45% w/v, about 1% w/v to about 40% w/v, about 2% w/v to about 35% w/v, about 5% w/v to about 30% w/v, about 10% w/v to about 25% w/v, or about 15% to about 20% w/v.

In some embodiments, the ophthalmic solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, greater than 40% w/v, or greater than 45% w/v, up to about 50% w/v.

In some embodiments, the ophthalmic solution contains one or more excipients or additives selected from a tonicity agent, buffering agent, wetting agent, viscosity enhancing agent, lubricating agent, chelating agent, and antioxidant. As discussed herein, in some embodiments, the contact lens solution contains a disinfecting agent, antiseptic agent, and/or a preservative, where appropriate.

In some embodiments, the ophthalmic solution is used in preventing, reducing the risk of, or treating a disorder or condition associated with wearing a contact lens. In some embodiments, a method of preventing, reducing the risk of, or treating a disorder or condition associated with wearing a contact lens comprises topically administering to an eye of a patient in need thereof a therapeutically effective amount of an ophthalmic solution comprising cyclodextrin, as described herein. In some embodiments, the ophthalmic solution is administered to the eye with the contact lens in contact with the eye. In some embodiments, the ophthalmic solution is administered to the eye without the contact lens in contact with the eye.

In some embodiments, the disorder or condition associated with wearing a contact lens for prevention or treatment is characterized by the presence of an inflammatory response or inflammatory condition. In some embodiments, the disorder or condition associated with wearing a contact lens is characterized by an abnormal oxidative stress reaction, particularly oxidative stress reaction characterized by lipid peroxidation. In some embodiments, the disorder or condition associated with wearing a contact lens for prevention or treatment is corneal edema, superficial keratitis, non-microbial associated red eye (contact lens acute red eye), endothelial polymegethism, Giant Papillary Conjunctivitis, corneal neovascularization, meibomitis, allergy aggravation, pingueculitis, ptosis, contact lens-induced peripheral ulcer, deep stromal corneal opacities, corneal hypoxia, epithelial microcysts, excess mucus production, or eye dryness, resulting from wearing contact lens.

### 4. DETAILED DESCRIPTION

The present disclosure relates to a contact lens storage solution containing cyclodextrin for preventing, reducing, or treating side effects or complications associated with wearing a contact lens and ophthalmic compositions containing cyclodextrin for preventing, reducing the risk of, or treating irritation, pain, and/or complications associated with wearing a contact lens. In some embodiments, the present disclosure provides a combination, for example a combination product, a contact lens package, or kit, comprising a contact lens in a contact lens solution comprising cyclodextrin, and an ophthalmic solution comprising cyclodextrin as prophylaxis for reducing the risk of, or as treatment for disorders or conditions associated with wearing a contact lens.

As used in this specification and the appended claims, the singular forms “a”, “an” and “the” include plural referents unless the context clearly indicates otherwise. Thus, for example, reference to “a compound” refers to more than one compound.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The foregoing general description, including the drawings, and the following detailed description are exemplary and explanatory only and are not restrictive of this disclosure.

The section headings used herein are for organizational purposes only and not to be construed as limiting the subject matter described.

### 4.1. Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings:

“Pharmaceutically active component” or “pharmaceutically active agent” refers to a pharmacophore, such as a drug or other therapeutic compound. In some embodiments, the “pharmaceutically active component” or “pharmaceutically active agent” is capable of forming inclusion complexes with a cyclodextrin.

“Treating” or “treatment” of a disease, disorder, or syndrome, as used herein, includes (i) preventing the disease, disorder, or syndrome from occurring in a subject, i.e., causing the clinical symptoms of the disease, disorder, or syndrome not to develop in a subject that may be exposed to or predisposed to the disease, disorder, or syndrome but does not yet experience or display symptoms of the disease, disorder, or syndrome; (ii) inhibiting the disease, disorder, or syndrome i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction, and the severity of the condition may be necessary, and is ascertainable by one of ordinary skill in the art.

“Prophylactic treatment” is a treatment administered to a subject who does not display signs or symptoms of a disease, pathology, or medical disorder, or displays only early signs or symptoms of a disease, pathology, or medical disorder, for the purpose of diminishing, preventing, or decreasing the risk of developing the disease, pathology, or medical disorder. A prophylactic treatment functions as a preventative treatment against a disease or disorder.

“Therapeutically effective amount” means any amount which, as compared to a corresponding subject who has not received such amount, results in improved treatment, healing, prevention, or amelioration of a disease or disorder, or a decrease in the rate of advancement of a disease or disorder, and also includes amounts effective to enhance normal physiological function.

“Effective amount” refers to that amount of a drug or pharmaceutical agent that will result in the desired biological or medical response that is being sought of a tissue, system, animal or human.

“Alkyl” refers to a noncyclic straight chain or branched, unsaturated or saturated hydrocarbon such as those containing from 1 to 10 carbon atoms, particularly 1 to 8 carbon atoms, more particularly 1 to 6 carbon atoms. Exemplary alkyls include, among others, methyl, ethyl, propyl, butyl, pentyl and hexyl.

“Pharmaceutically acceptable” as used herein refers to materials or substances that are generally not toxic or injurious to a subject.

“Additive” in the context of a pharmaceutical composition is intended to include any pharmaceutically acceptable carrier, diluent or excipient, particularly a carrier, diluent, or excipient suitable for ophthalmic use.

“Excipient” refers to an ingredient or component that provides one or more of bulk, imparts satisfactory processing characteristics, helps control the dissolution rate, or otherwise gives additional desirable characteristics to the compositions. Included within this term, inter alia, are compounds well known to those of ordinary skill in the art, as described, for example, in the Handbook of Pharmaceutical Excipients, 4th Ed., American Pharmaceutical Association, Washington, D.C. and Pharmaceutical Press, London, England, 2003), incorporated herein by reference in its entirety.

“Opthalmically acceptable” refers to a composition that is suitable for use on the eye of a patient, for example a composition that does not trigger pain and/or abnormal secretion of tears. An ophthalmically acceptable excipient refers to an ingredient or component that does not result in pain and/or abnormal secretion of tears when administered to the eye.

“Substantially free” of a pharmaceutically active agent or component or equivalents thereof refers to at least a level of a pharmaceutically active agent or component which is below the effective level (e.g., concentration) of the pharmaceutically active agent or component.

“About” or “approximately” refers to an acceptable error range for the particular value as determined by one of ordinary skill in the art, which will depend in part on how the value is measured or determined, i.e., the limitations of the measurement system. For example, “about” can mean a range of up to 20%, preferably up to 10%, more preferably up to 5%, and more preferably still up to 1% of a given value.

### 4.2. Contact Lens Solutions and Contact Lens Combination Products

In one aspect, the present disclosure provides a contact lens solution for storing contact lenses, in particular in storing or immersing contact lenses prior to placement on the eye. In some embodiments, the contact lens solution comprises a cyclodextrin. In some embodiments, the contact lens solution is prepared as an ophthalmically acceptable contact lens solution.

In some embodiments, the contact lens solution comprising a cyclodextrin is substantially free of a disinfecting agent, antiseptic agent, and/or a preservative. In some embodiments, the contact lens solution containing cyclodextrin does not contain any disinfecting agent, antiseptic agent, and/or a preservative.

As used herein, the term “substantially free of a disinfecting agent, antiseptic agent and/or a preservative” refers to a concentration or amount of a disinfecting agent, antiseptic agent and/or preservative that is ineffective in disinfection, antiseptic effects and/or a preservative effects. In some embodiments, substantially free of a disinfecting agent, antiseptic agent and/or a preservative refers to less than 0.005% w/v, less than 0.001% w/v, less than 0.0005% w/v, less than 0.0001% w/v, or less than 0.00005% w/v of a disinfecting agent, antiseptic agent and/or a preservative.

In some embodiments, the contact lens solution comprising a cyclodextrin contains a disinfecting agent, antiseptic agent and/or a preservative. In some embodiments, the contact lens solution comprising a cyclodextrin contains a disinfecting agent, antiseptic agent and/or a preservative, particularly when provided together as a combination product, for example in a package or kit, for example with an ophthalmic solution containing cyclodextrin, as further described herein.

In some embodiments of the contact lens solution, the cyclodextrin is substantially free of inclusion complexes, particularly substantially free of inclusion complexes with a pharmaceutically active agent. In some embodiments, the contact lens solution contains cyclodextrin which is substantially free of a pharmaceutically active agent capable of forming an inclusion complex with the cyclodextrin. In some embodiments, the contact lens solution contains cyclodextrin as the sole or only active agent in the contact lens solution.

In some embodiments, the contact lens solution contains cyclodextrin at a concentration sufficient to coat the contact lens or provide a film or layer of cyclodextrin when placed onto the eye. In some embodiments, the concentration of cyclodextrin is sufficient to provide a mucomimetic layer or film of cyclodextrin between the eye surface and the contact lens when the contact lens is placed onto the eye.

In some embodiments, the concentration of cyclodextrin in the contact lens solution is effective to bind a biological mediator or effector of a disorder or condition associated with wearing a contact lens. In some embodiments, the biological mediator is an allergen, prostaglandin, and/or a toxic aldehyde, for example malondialdehyde or 4-hydroxynonenal.

Generally, cyclodextrins are compounds composed of sugar molecules bound together in a ring to form a structure with an internal cavity that can form inclusion complexes with other compounds. In some embodiments, the sugar molecules of the cyclodextrin are composed of α-D-glucopyranosyl units connected via α(1,4) linkages to form a torus like structure, where the size of the internal cavity is determined, in part, by the number of glucopyranose units. The number of sugar units can range from 5 to 32 or more. Naturally occurring cyclodextrins include, among others, α-cyclodextrin (6 glucopyranosyl units), β-cyclodextrin (7 glucopyranosyl units) and γ-cyclodextrin (8 glucopyranosyl units). Unless specifically described otherwise, the term “cyclodextrin” includes derivatives of cyclodextrin compounds.

In some embodiments, the cyclodextrin for the contact lens solution is α-cyclodextrin, β-cyclodextrin, γ-cyclodextrin, derivatives thereof, or combinations thereof. The cc-, β-, and γ-cyclodextrins are naturally occurring cyclodextrins, but numerous derivatives of these cyclodextrins have been made. In some embodiments, cyclodextrins derivatives include, among others, maltosyl, glucosyl, and maltotriosyl derivatives of β- and γ-cyclodextrins (see, e.g., U.S. Pat. No. 5,024,998, incorporated herein by reference). Other useful cyclodextrins have been synthesized by modification of the hydroxyl groups on the glycosyl units, for example by amination, esterification or etherification. These modifications to the cyclodextrin can result in changes to the cavity volume, solubility, and differential reactivity with guest molecules.

In some embodiments, the cyclodextrin in the contact lens solution is selected from α-cyclodextrin, β-cyclodextrin, γ-cyclodextrin, derivatives thereof, and combinations thereof. In particular, the cyclodextrin is selected from β-cyclodextrin, γ-cyclodextrin, derivatives thereof, and combinations thereof.

In some embodiments, the cyclodextrin is selected from carboxyalkyl cyclodextrin, hydroxyalkyl cyclodextrin, sulfoalkylether cyclodextrin, and alkyl cyclodextrin. In various embodiments, the alkyl group in the cyclodextrin is methyl, ethyl, propyl, butyl, pentyl, or hexyl.

In some embodiments, the cyclodextrin is α-cyclodextrin or a derivative thereof. In some embodiments, the α-cyclodextrin or a derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof. In some embodiments, the alkyl group in the α-cyclodextrin derivative is methyl, ethyl, propyl, butyl, pentyl or hexyl.

In some embodiments, the cyclodextrin is β-cyclodextrin or a derivative thereof. In some embodiments, the β-cyclodextrin or derivative thereof is selected from carboxyalkyl-β-cyclodextrin, hydroxyalkyl-β-cyclodextrin, sulfoalkylether-β-cyclodextrin, alkyl-β-cyclodextrin, and combinations thereof. In some embodiments, the alkyl group in the β-cyclodextrin derivative is methyl, ethyl, propyl, butyl, pentyl or hexyl.

In some embodiments, the β-cyclodextrin or a derivative thereof is hydroxyalkyl-β-cyclodextrin or sulfoalkylether-β-cyclodextrin. In some embodiments, the hydroxyalkyl-β-cyclodextrin is hydroxypropyl-β-cyclodextrin. In some embodiments, the sulfoalkylether-β-cyclodextrin is sulfobutylether-β-cyclodextrin. In some embodiments, β-cyclodextrin or a derivative thereof is alkyl-β-cyclodextrin, in particular methyl-β-cyclodextrin. In some embodiments using methyl-β-cyclodextrin, the β-cyclodextrin is randomly methylated β-cyclodextrin.

In some embodiments, the cyclodextrin is γ-cyclodextrin or a derivative thereof. In some embodiments, the γ-cyclodextrin or derivative thereof is selected from carboxyalkyl-γ-cyclodextrin, hydroxyalkyl-γ-cyclodextrin, sulfoalkylether-γ-cyclodextrin, and alkyl-γ-cyclodextrin. In some embodiments, the alkyl group in the γ-cyclodextrin derivative is methyl, ethyl, propyl, butyl, pentyl, or hexyl. In some embodiments, the γ-cyclodextrin or derivative thereof is hydroxyalkyl-γ-cyclodextrin or sulfoalkylether-γ-cyclodextrin. In some embodiments, the hydroxyalkyl-γ-cyclodextrin is hydroxypropyl-γ-cyclodextrin, such as 2-hydroxypropyl-γ-cyclodextrin. In some embodiments, the γ-cyclodextrin or derivative thereof.

In some embodiments, the cyclodextrin is 6^{A},6^{B},6^{C},6^{D},6^{E},6^{F},6^{G},6^{H}-Octakis-S -(2-carboxyethyl)-6^{A},6^{B},6^{C},6^{D},6^{E},6^{F},6^{G},6^{H}-octathio-γ-cyclodextrin (i.e., S-2-carboxyethyl-octathio-γ-cyclodextrin, also referred to as sugammadex) and/or 6^{A},6^{B},6^{C},6^{D},6^{E},6^{F},6^{G}-Heptakis-S -(2-carboxyethyl)-6^{A},6^{B},6^{C},6^{D} ,6^{E},6^{F},6^{G}-heptathio-γ-cyclodextrin (i.e., S-2-carboxyethyl-heptathio-γ-cyclodextrin). Thus, in some embodiments, the cyclodextrin is 2-carboxyethyl-octathio-γ-cyclodextrin or 2-carboxyethyl-heptathio-γ-cyclodextrin.

In some embodiments, the cyclodextrin contact lens solution contains a plurality of different cyclodextrins. In some embodiments, the contact lens solution contains a combination or mixture of different cyclodextrins. In some embodiments, the contact lens solution contains a first cyclodextrin and a second cyclodextrin, where the first cyclodextrin is different from the first cyclodextrin. In some embodiments, mixtures of cyclodextrins can be a combination of: α-cyclodextrin and β-cyclodextrin, including combinations of α-cyclodextrin and β-cyclodextrin derivatives; α-cyclodextrin and γ-cyclodextrin, including combinations of α-cyclodextrin and γ-cyclodextrin derivatives; β-cyclodextrin and γ-cyclodextrin, including combinations of β-cyclodextrin and γ-cyclodextrin derivatives; or α-cyclodextrin, β-cyclodextrin, and γ-cyclodextrin, including combinations of α-cyclodextrin, β-cyclodextrin, and γ-cyclodextrin derivatives.

In some embodiments, various salts of the cyclodextrin or salts of the cyclodextrin derivative can be used in the contact lens solution. In some embodiments, the salts are pharmaceutically acceptable salt(s), which refers to those salts of compounds i.e., cyclodextrin, that are safe and effective for use in mammals and that possess the desired biological activity. Pharmaceutically acceptable salts include salts of acidic or basic groups present in the cyclodextrins. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, and p-toluenesulfonate salts. Suitable base salts include, but are not limited to, aluminum, calcium, lithium, magnesium, potassium, sodium, zinc, and diethanolamine salts. In some embodiments, the cyclodextrin is in the form of a sodium or potassium salt. In some embodiments, the cyclodextrin is in the form an ophthalmically acceptable salt. Guidance on suitable pharmaceutically acceptable salts, ophthalmically acceptable salts, and their application to drug formulations can be found in various references, such as Remington's Pharmaceutical Sciences, 17th Ed., Mack Publishing Company, Easton, Pa., 1985, and Berge, et al., 1977, “Pharmaceutical Salts,” J Pharm Sci. 66:1-19, both of which are incorporated herein by reference.

In some embodiments, the contact lens solution contains cyclodextrin at a concentration of about 0.1% w/v to about 50% w/v, about 0.5% w/v to about 45% w/v, about 1% w/v to about 40% w/v, about 2% w/v to about 35% w/v, about 5% w/v to about 30% w/v, about 10% w/v to about 25% w/v, or about 15% to about 20% w/v.

In some embodiments, the contact lens solution contains cyclodextrin at a concentration of about 0.1% w/v, about 0.2% w/v, about 0.3%, w/v, about 0.4% w/v, about 0.5% w/v, about 0.6% w/v, about 0.7% w/v, about 0.8% w/v, about 0.9% w/v, about 1% w/v, about 1.5% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 35% w/v, about 40% w/v, about 45% w/v, or about 50% w/v.

In some embodiments, the contact lens solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

In some embodiments, the contact lens solution contains cyclodextrin at a concentration greater than 5% w/v, for example, about 6% w/v to about 50% w/v, about 7% w/v to about 50% w/v, about 8% w/v to about 50% w/v, about 9% w/v to about 50% w/v, about 10% w/v to about 50% w/v, about 15% w/v to about 45% w/v, about 20% w/v to about 40% w/v, or about 25% w/v to about 35% w/v.

In some embodiments, the contact lens solution contains cyclodextrin at a concentration of about 15% w/v to about 50% w/v, about 20% w/v to about 50% w/v, or about 25% w/v to about 50% w/v, about 30% w/v to about 50% w/v, about 35% w/v to about 50% w/v, or about 40% w/v to about 50% w/v.

In some embodiments, the contact lens solution contains cyclodextrin at a concentration of about 6% w/v to about 40% w/v, about 7% w/v to about 40% w/v, about 8% w/v to about 40% w/v, about 9% w/v to about 40% w/v, about 10% w/v to about 40% w/v, about 15% w/v to about 40% w/v, about 20% w/v to about 40% w/v, or about 25% w/v to about 40% w/v, about 30% w/v to about 40% w/v, or about 35% w/v to about 40% w/v.

In some embodiments, the contact lens solution contains cyclodextrin at a concentration of about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 25% w/v, about 30% w/v, about 35% w/v, about 40% w/v, about 45% w/v, or about 50% w/v.

In some embodiments, the contact lens solution contains cyclodextrin at a concentration greater than 10% w/v, for example, about 11% w/v to about 50% w/v, about 12% w/v to about 50% w/v, about 13% w/v to about 50% w/v, about 14% w/v to about 50% w/v, about 15% w/v to about 50% w/v, about 16% w/v to about 50% w/v, about 17% w/v to about 50% w/v, about 18% w/v to about 50% w/v, about 19% w/v to about 50% w/v, about 20% w/v to about 50% w/v, about 25% w/v to about 45% w/v, or about 30% w/v to about 40% w/v.

In some embodiments, the contact lens solution contains cyclodextrin at a concentration greater than 10% w/v, for example, at about 11% w/v, about 12% w/v, about 13% w/v, about 14% w/v, about 15% w/v, about 16% w/v, about 17% w/v, about 18% w/v, about 19% w/v, about 20% w/v, about 25% w/v, about 30% w/v, about 35% w/v, about 40% w/v, about 45% w/v, or about 50% w/v.

In some embodiments, the cyclodextrin is present in the specified concentrations above. In some embodiments, the cyclodextrin is a α-cyclodextrin, β-cyclodextrin, γ-cyclodextrin, derivatives thereof, or combinations thereof at the specified concentrations above. In some embodiments, the β-cyclodextrin or derivative thereof is hydroxyalkyl-β-cyclodextrin or sulfoalkylether-β-cyclodextrin, particularly hydroxypropyl-β-cyclodextrin or sulfobutylether-β-cyclodextrin, at the specified concentrations above. In some embodiments, the cyclodextrin is a methyl-β-cyclodextrin, in particular randomly methylated-β-cyclodextrin, at the specified concentrations above. In some embodiments, the γ-cyclodextrin or derivative thereof is hydroxyalkyl-γ-cyclodextrin or sulfoalkylether-γ-cyclodextrin, particularly hydroxypropyl-γ-cyclodextrin or sulfobutylether-γ-cyclodextrin at the specified concentrations above. In some embodiments where the contact lens solution contains a mixture of cyclodextrins, the concentrations described above are for the total cyclodextrin in the contact lens solution.

In some embodiments, the cyclodextrin contact lens solution contains in addition to the cyclodextrin, one or more ophthalmic pharmaceutically acceptable additive or excipient selected from a tonicity agent, buffering agent, wetting agent, viscosity enhancing agent, lubricating agent, chelating agent, antioxidant, pH adjusting agent, solubilizing agent, surfactant, chelating agent, emulsifying agent, suspending agent, and stabilizing agent. In some embodiments, the contact lens solution contains a disinfecting agent, antiseptic agent, and/or a preservative, where appropriate. In some embodiments, the additive or excipient is ophthalmically acceptable.

In some embodiments, the cyclodextrin contact lens solution can have one or more tonicity agents, which can be used to adjust the tonicity of the contact lens solution, for example to reduce irritation and make it compatible when placing the contact lens on the eye, for example, to the tonicity of natural tears. Suitable tonicity agents include, by way of example and not limitation, dextrans (e.g., dextran 40 or 70), dextrose, glycerin, potassium chloride, propylene glycol, and sodium chloride. Equivalent amounts of one or more salts made up of cations, for example, such as potassium, ammonium and anions such as chloride, citrate, ascorbate, borate, phosphate, bicarbonate, sulfate, thiosulfate, bisulfate, the salts sodium bisulfate and ammonium sulfate, can also be used. The amount of tonicity agent will vary, depending on the particular agent to be added. In general, however, the contact lens solution can have a tonicity agent in an amount sufficient to cause the final composition to have an ophthalmically acceptable osmolarity. In some embodiments, the cyclodextrin compositions have an osmolarity of about 200 to about 1000 mOsm/L or about 200 to about 500 mOsm/L, or any specific value within said ranges (e.g., 200 mOsm/L, 210 mOsm/L, 220 mOsm/L, 230 mOsm/L, 240 mOsm/L, 250 mOsm/L, 260 mOsm/L, 270 mOsm/L, 280 mOsm/L, 290 mOsm/L, 300 mOsm/L, 310 mOsm/L, 320 mOsm/L, 330 mOsm/L, 340 mOsm/L, 350 mOsm/L, 360 mOsm/L, 370 mOsm/L, 380 mOsm/L, 390 mOsm/L or 400 mOsm/L). In a particular embodiment, the ophthalmic formulations are adjusted with a tonicity agent to an osmolarity ranging from about 250 to about 450 mOsm/L, or about 250 to about 350 mOsm/L.

In some embodiments, the cyclodextrin contact lens solution contains one or more buffering agents for adjusting and/or maintaining the pH of the contact lens solution at a specified pH range. Generally, buffer capacity should be large enough to maintain the product pH for a reasonably long shelf-life but also low enough to allow rapid readjustment of the product to physiologic pH upon placement of the contact lens to the eye. Generally, buffer capacities of from about 0.01 to 0.1 can be used for contact lens solutions, particularly at concentrations that provide sufficient buffering capacity and minimizes adverse effects e.g., irritation, to the eye. Exemplary buffering agents include, by way of example and not limitation, various salts (e.g., sodium, potassium, etc.), acids or bases, where appropriate, of the following agents, including, among others, acetate, borate, phosphate, bicarbonate, carbonate, citrate, tetraborate, biphosphate, tromethamine, hydroxyethyl morpholine, and THAM (trishydroxymethylamino-methane). In some embodiments, the buffering agent can be present from about 0.01 mM to about 100 mM, about 0.05 mM to about 100 mM, about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM. In some embodiments, the buffering agent can be present at about 0.01 mM, about 0.05 mM, about 0.1 mM, about 0.2 mM, about 0.5 mM, about 1 mM, about 5 mM, about 10 mM, about 20 mM, about 30 mM, about 40 mM, about 50 mM, or about 100 mM.

In some embodiments, an exemplary buffering agent is phosphate, particularly sodium phosphate, which can be prepared by standard procedures, for example by mixing appropriate amounts of one or more monobasic phosphates, dibasic phosphates, and the like. In particular, useful phosphate buffers are prepared from phosphate salts of alkali and/or alkaline earth metals, such as sodium or potassium phosphate, including sodium monobasic phosphate, sodium dibasic phosphate, potassium monobasic phosphate, and potassium dibasic phosphate. In some embodiments, the phosphate buffer can be present from about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM. In some embodiments, the phosphate buffer can be present at about 0.5 mM, about 1 mM, about 5 mM, about 10 mM, about 20 mM, about 30 mM, about 40 mM, about 50 mM, or about 100 mM.

In some embodiments, the cyclodextrin contact lens solution contains one or more wetting agents. Generally, wetting agents are hydrophilic polymers, including, by way of example and not limitation, polysorbate 20 and 80, poloxamer 282, and tyloxapol. In some embodiments, wetting agents also include, among others, cellulose based polymers, such as hydroxypropylmethylcellulose (HPMC) and carboxymethylcellulose (CMC); polyvinylpyrrolidone; and polyvinyl alcohol. In some embodiments, the concentration of wetting agent, such as HPMC, ranges from about 0.1% to about 2% w/v, about 0.5% to about 1% w/v, or any specific value within the ranges. In some embodiments, the concentration of wetting agent, such as HPMC, ranges from about 0.1% to about 1.0% w/v, or any specific value within said range (e.g., 0.1-0.2%, 0.2-0.3%, 0.3-0.4%, 0.4-0.5%, 0.5-0.6%, 0.6-0.7%, 0.7-0.8%, 0.8-0.9%, 0.9-1.0%; about 0.2%, about 0.21%, about 0.22%, about 0.23%, about 0.24%, about 0.25%, about 0.26%, about 0.27%, about 0.28%, about 0.29%, about 0.30%, about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

In some embodiments, the cyclodextrin contact lens solution contains one or more viscosity enhancing agents. The viscosity enhancing agent typically enhances the viscosity of the contact lens solution to increase retention time of the solution on the contact lens and/or the eye, and in some instances, to provide a protective layer when the contact lens is placed on the eye. Viscosity enhancing agents include, among others, hyaluronate, hyaluronic acid or pharmaceutically acceptable salt thereof; cross-linked hyaluronic acid; carbopol gels, dextran (e.g., dextran 40, molecular weight of 40,000 Daltons; dextran 70, molecular weight of 70,000 Daltons), gelatin, glycerin, carboxymethyl cellulose, hydroxyethyl cellulose, hydroxypropyl methylcellulose, methylcellulose, ethylcellulose, polyethylene glycol, poloxamer 407, polysorbate 80, propylene glycol, glycerol, polyvinyl alcohol, and polyvinylpyrrolidone (povidone), in various molecular weights and in various compatible combinations. In some embodiments, the contact lens solution has a viscosity that ranges from about 10 to about 150 centipoise (cP), about 15 to about 120 cP, about 20 to about 90 cP (or any specific value within said ranges). In some embodiments, the contact lens solution comprising the cyclodextrin has a viscosity that ranges from about 15 cP to about 30 cP, or any specific value within the range (i.e., about 15 cP, about 16 cP, about 17 cP, about 18 cP, about 19 cP, about 20 cP, about 20 cP, about 22 cP, about 23 cP, about 24 cP, about 25 cP, about 26 cP, about 27 cP, about 28 cP, about 29 cP, about 30 cP). In some embodiments, the contact lens solution comprising the cyclodextrin has a viscosity that ranges from about 70 cP to about 90 cP, or any specific value within said range (i.e., about 70 cP, about 71 cP, about 72 cP, about 73 cP, about 74 cP, about 75 cP, about 76 cP, about 77 cP, about 78 cP, about 79 cP, about 80 cP, about 81 cP, about 82 cP, about 83 cP, about 84 cP, about 85 cP, about 86 cP, about 87 cP, about 88 cP, about 89 cP or about 90 cP). In particular, a viscosity of from about 25 to about 50 cP are suitable for ophthalmic solutions. The viscosity enhancing component is present in an amount effective in providing the desired viscosity to the composition.

In some embodiments, the amount of the viscosity enhancing agent is based on the agent used, and can be in general be an amount of about 0.05 w/v to 30% w/v. In some embodiments, the concentration of viscosity enhancing agent is about 0.05 w/v to 30% w/v, about 0.1% w/v to about 25% w/v, about 0.25% w/v to about 15% w/v, about 0.5% w/v to about 15% w/v, about 0.75% w/v to about 10% w/v, or about 1.0% w/v to about 5% w/v.

In some embodiments, the amount of the viscosity enhancing agent in the contact lens solution is about 0.05% w/v to about 1.5% w/v, about 0.05% w/v to about 0.5% w/v, about 0.1% w/v to about 3.0% w/v, about 0.1% w/v to about 1.5% w/v, about 0.1% w/v to about 1.0% w/v, about 0.5% w/v to about 1% w/v, about 0.5% w/v to about 2.5% w/v, about 1.0% w/v to about 3.0% w/, about 1.0% w/v to about 1.5% w/v, about 1.0% w/v to about 1.25% w/v, about 1.25% w/v to about 1.5% w/v, or about 1.5% w/v to about 3.0% w/v.

In some embodiments, the amount of the viscosity enhancing agent in the contact lens solution is about 0.1% w/v, about 0.25% w/v, about 0.5% w/v, about 0.75% w/v, about 1.0% w/v, about 1.1% w/v, about 1.15% w/v, about 1.20% w/v, about 1.25% w/v, about 1.30% w/v, about 1.35% w/v, about 1.40% w/v, about 1.45% w/v, about 1.5% w/v, about 2% w/v, about 3% w/v, about 4% w/v, about 5% w/v, about 10% w/v, about 15% w/v, about 20% w/v, about 25% w/v, or about 30% w/v.

In some embodiments, the molecular weight of a viscosity enhancing agent when polymeric is about 500,000 to about 5×10⁶ Daltons; about 500,000 Daltons to about 3×10⁶ Daltons; about 500,000 to about 2×10⁶ Daltons; about 500,000 to about 1×10⁶ Daltons; about 500,000 to about 2×10⁶ Daltons; about 1×10⁶ Daltons to about 3×10⁶ Daltons; about 1×10⁶ Daltons to about 2.5×10⁶ Daltons; about 1×10⁶ Daltons to about 2×10⁶ Daltons; or about 1.2×10⁶ Daltons to about 1.8×10⁶ Daltons. In some embodiments, the molecular weight is the number average molecular weight, and in other embodiments the molecular weight is the weight average molecular weight. Preferably the molecular weight is the number average molecular weight. In some embodiments, the viscosity enhancing agent comprises hyaluronate, hyaluronic acid or pharmaceutically acceptable salt thereof.

In some embodiments, the cyclodextrin contact lens solution contains one or more lubricating agents. In some embodiments, the lubricants can approximate the consistency of endogenous tears and aid in natural tear build-up when the contact lens is applied to the eye. Lubricating agents can include non-phospholipid and phospholipid-based agents. In some embodiments, lubricants that are non-phospholipid based include, but are not limited to, propylene glycol; ethylene glycol; polyethylene glycol; hydroxypropylmethylcellulose; carboxymethylcellulose; hydroxypropylcellulose; dextrans, such as, dextran 70; water soluble proteins, such as gelatin; vinyl polymers, such as polyvinyl alcohol, polyvinylpyrrolidone, povidone; petrolatum; mineral oil; and carbomers, such as, carbomer 934P, carbomer 941, carbomer 940, and carbomer 974P. Non-phospholipid lubricants can also include compatible mixtures of any of the foregoing agents.

In some embodiments, the cyclodextrin contact lens solution contains one or more chelating agents. In some embodiments, the chelating agent is selected from ethylenediaminetetracetic acid (EDTA), ethylene glycol-bis(β-aminoethyl ether)-N,N,N′,N′-tetraacetic acid (EGTA), dihydroxy ethyl glycine, citric acid, and tartaric acid.

In some embodiments, the cyclodextrin contact lens solution contains one or more disinfecting agent and/or antiseptic agents, where appropriate and compatible with contact lens material. In some embodiments, the disinfecting agent and/or antiseptic agent is selected from boric acid, hexamidine di-isetionate, polyaminopropyl-biguanide, polyhexamide, polyyquaternium, myristamidopropyl dimethylamine, and thimerosol.

In some embodiments, the cyclodextrin contact lens solution can have one or more preservatives, for example, to extend shelf life or limit bacterial growth in the solutions during storage as well as when administered therapeutically onto the eye. Preservatives that can be used, include, among others, benzalkonium chloride, benzethonium chloride, benzododecinium bromide, cetylpyridinium chloride, chlorobutanol, thimerosol, phenylmercuric nitrate, phenylmercuric acetate, methyl/propylparabens, phenylethyl alcohol, sodium benzoate, sodium propionate, sorbic acid, and sodium perborate. The amount of preservative in the solution can be a level that enhances the shelf life, limits bacterial growth, or otherwise preserves the ocular solution, with minimal toxicity to the eye tissues (see, e.g., The United States Pharmacopeia, 22nd rev., and The National Formulary, 17th Ed. Rockville, Md.). Concentration of preservative suitable for use in the contact lens solution can be determined by the person skilled in the art. In some embodiments, the preservatives can be used at an amount of from about 0.001% w/v to about 1.0% w/v. For example, the preservative is present from about 0.005% w/v to about 0.05% w/v, 0.005% w/v to about 0.04% w/v, 0.01% w/v to about 0.03% w/v, 0.01% w/v to about 0.02% w/v, or from about 0.01% w/v to about 0.015% w/v. In some embodiments, the amount of preservative can be about 0.005% w/v, about 0.01% w/v, about 0.012% w/v, about 0.014% w/v, about 0.016% w/v, about 0.018% w/v, about 0.02% w/v, about 0.03% w/v, about 0.04% w/v, or about 0.05% w/v. In some embodiments, no preservatives are used in the compositions.

In some embodiments, the cyclodextrin contact lens solution can include one or more antioxidants. Suitable antioxidants, include, by way of example and not limitation, EDTA (e.g., disodium EDTA), sodium bisulphite, sodium metabisulphite, sodium thiosulfate, thiourea, and alpha-tocopherol.

In some embodiments, the cyclodextrin contact lens solution can include a pH adjusting agent. In some embodiments, pH adjusting agents include, by way of example and not limitation, various salts (e.g., sodium, potassium, etc.), acids or bases, where appropriate, of the following agents, including, among others, chloride, acetate, borate, phosphate, bicarbonate, carbonate, citrate, tetraborate, biphosphate, tromethamine, hydroxyethyl morpholine, and THAM (trishydroxymethylamino-methane).

In some embodiments, the cyclodextrin contact lens solution can include a solubilizing agent. In some embodiments, the solubilizing agent that can be used include, among others, surfactant or emulsifying agent.

In some embodiments, the cyclodextrin contact lens solution can include a surfactant. In some embodiments, the surfactant is a non-ionic surfactant. In some embodiments, the surfactant is selected from, among others, sorbitan esters such as and not limited to Spans and mixtures of the partial esters of sorbitol and its mono- and di-anhydrides with oleic acid; polysorbates such as and not limited to polysorbate 20 and 80; and poloxamers, such as and not limited to, poloxamer 282, pluronics and tyloxapol

In some embodiments, the cyclodextrin contact lens solution can have an emulsifying agent. In some embodiments, the emulsifying agent can be oil in water where the oil phase is a medium chain triglyceride or one or more oils selected from the group consisting of olive, soy, corn, mineral, cottonseed, safflower, and sesame oil.

In some embodiments, the cyclodextrin contact lens solution can have a stabilizing agent, suspending agent and viscosity modifying agent. These include, but are not limited to, among others, cellulose based polymers, such as hydroxyethylcellulose, hydroxypropylmethylcellulose (HPMC) and carboxymethylcellulose (CMC); polyvinylpyrrolidone; and polyvinyl alcohol.

In various embodiments, the pH of the cyclodextrin contact lens solution can be within 1.0 to 1.5 pH units from physiological pH, particularly the physiological pH in the external environment of the eye. The pH of human tears is approximately pH 7.4. Hence, the pH of the solution can be about 1.0 to 1.5 pH units above or below pH 7.4. In some embodiments, the pH of the contact lens solution is from about pH 6.0 to about pH 8.5. In some embodiments, the pH of the contact lens solution is from about pH 6.0 to about pH 8.0. In some embodiments, the pH of the contact lens solution is from about 6.5 to about 8.0. In some embodiments, the pH of the contact lens solution is from about 7.0 to about 8.0. In some embodiments, the pH of the contact lens solution is from about 7.0 to about 7.5. In some embodiments, the pH of the contact lens solution is about 6.5, about 7, about 7.5, about 8, or about 8.5. A person of skill in the art can select a pH that balances stability of the cyclodextrin composition and the tolerability of the contact lens and the eye to differences in pH from the natural condition. As is well known in the art, the pH of the solution can be adjusted by using appropriate buffering agents and/or with an appropriate base (e.g., sodium hydroxide) or acid (e.g., hydrochloric acid).

In some embodiments, the contact lens solution comprises a cyclodextrin, and at least a viscosity enhancing agent, and a buffering agent. In some embodiments, the contact lens solution comprises a cyclodextrin, and at least a viscosity enhancing agent, a buffering agent, and optionally a disinfecting agent, antiseptic agent and/or a preservative. In some embodiments, the contact lens solution is substantially free of a disinfecting agent, antiseptic agent, and/or a preservative.

In some embodiments, the contact lens solution comprises a cyclodextrin, and at least a viscosity enhancing agent, a buffering agent, and a tonicity agent. In some embodiments, the contact lens solution comprises a cyclodextrin, and at least a viscosity enhancing agent, a buffering agent, a tonicity agent, and optionally a disinfecting agent, antiseptic agent, and/or a preservative. In some embodiments, the contact lens solution is substantially free of a disinfecting agent, antiseptic agent, and/or a preservative.

In some embodiments, the contact lens solution comprises a cyclodextrin, and at least a wetting agent, and a buffering agent. In some embodiments, the contact lens solution comprises a cyclodextrin, and at least a wetting agent, a buffering agent, and optionally a disinfecting agent, antiseptic agent and/or a preservative. In some embodiments, the contact lens solution is substantially free of a disinfecting agent, antiseptic agent and/or a preservative.

In some embodiments, the contact lens solution comprises a cyclodextrin, and at least a wetting agent, a buffering agent, and a tonicity agent. In some embodiments, the contact lens solution comprises a cyclodextrin, and at least a wetting agent, a buffering agent, a tonicity agent, and optionally a disinfecting agent, antiseptic agent, and/or a preservative. In some embodiments, the contact lens solution is substantially free of a disinfecting agent, antiseptic agent, and/or a preservative.

In some embodiments, the contact lens solution comprises a cyclodextrin, and at least a viscosity enhancing agent and/or a wetting agent, and a buffering agent. In some embodiments, the contact lens solution comprises a cyclodextrin, and at least a viscosity enhancing agent and/or a wetting agent, a buffering agent, and optionally a disinfecting agent, antiseptic agent and/or a preservative. In some embodiments, the contact lens solution is substantially free of a disinfecting agent, antiseptic agent, and/or a preservative.

In some embodiments, the contact lens solution comprises a cyclodextrin, and at least a viscosity enhancing agent and/or a wetting agent, a buffering agent, and a tonicity agent. In some embodiments, the contact lens solution comprises a cyclodextrin, and at least a viscosity enhancing agent and/or a wetting agent, a buffering agent, a tonicity agent, and optionally a disinfecting agent, antiseptic agent and/or a preservative. In some embodiments, the contact lens solution is substantially free of a disinfecting agent, antiseptic agent, and/or a preservative.

In some embodiments, the contact lens solution comprises a cyclodextrin, and at least a lubricating agent, and a buffering agent. In some embodiments, the contact lens solution comprises a cyclodextrin, and at least a lubricating agent, a buffering agent, and optionally a disinfecting agent, antiseptic agent and/or a preservative. In some embodiments, the contact lens solution is substantially free of a disinfecting agent, antiseptic agent and/or a preservative.

In some embodiments, the contact lens solution comprises a cyclodextrin, and at least a lubricating agent, a buffering agent, and a tonicity agent. In some embodiments, the contact lens solution comprises a cyclodextrin, and at least a lubricating agent, a buffering agent, a tonicity agent, and optionally a disinfecting agent, antiseptic agent and/or a preservative. In some embodiments, the contact lens solution is substantially free of a disinfecting agent, antiseptic agent and/or a preservative.

In some embodiments, the contact lens solution is provided as a combination, for example as a combination product, a contact lens package, or a kit, comprising: (a) a contact lens in a first contact lens solution comprising a cyclodextrin, wherein the first contact lens solution contains a disinfecting agent, antiseptic agent and/or a preservative, and (b) a second contact lens solution comprising a cyclodextrin, wherein the second contact lens solution is substantially free of disinfecting agent, antiseptic agent and/or a preservative.

In some embodiments, the contact lens solution is provided as a combination, for example as a combination product, contact lens package, or a kit, comprising: (a) a contact lens in a first contact lens solution comprising a cyclodextrin, wherein the first contact solution is substantially free of a disinfecting agent, antiseptic agent, and/or preservative, and (b) a second contact lens solution comprising a cyclodextrin, wherein the second contact lens solution is substantially free of disinfecting agent, antiseptic agent, and/or a preservative.

In some embodiments, the second contact lens solution is provided in a separate container or dispenser, such as a single use container. In some embodiments, the second contact lens solution is used to rinse the contact lens after removal from the first contact lens solution to remove or dilute out the disinfecting agent, antiseptic agent and/or a preservative, before placing the contact lens on the eye.

In some embodiments, a contact lens package comprises: (a) contact lens case for holding one or more contact lenses, a contact lens positioned in the contact lens case, and a first contact lens solution comprising a cyclodextrin, wherein the contact lens is in the first contact lens solution; and (b) a second contact lens solution in a separate container or dispenser, wherein the second contact lens solution contains cyclodextrin and is substantially free of a disinfecting agent, antiseptic agent and/or preservative. In some embodiments, the first contact lens solution contains a disinfecting agent, antiseptic agent and/or preservative. In some embodiments, the first contact lens solution is substantially free of a disinfecting agent, antiseptic agent and/or preservative.

In some embodiments, the first contact lens solution and the second contact lens solution have a composition described above. In some embodiments, the first contact lens solution and the second contact lens solution are the same composition, other than presence of a disinfecting agent, antiseptic agent, and/or preservative. In some embodiments, the first contact lens solution and the second contact lens solution are different compositions, in particular different type and/or concentration of cyclodextrin.

In some embodiments, the contact lens case comprises a first compartment or chamber for storage of a contact lens, and a second compartment or chamber which can receive a contact lens and a contact lens solution. In some embodiments, the contact lens case comprises an upper part and a lower part, wherein the upper part is connected to the lower part by a hinge, and the lower part is configured to have one or more compartments or chambers capable of receiving a contact lens, wherein various solutions can be placed into the compartment or chamber, and the upper part moves relative to the lower part and forms a lid when the upper part and the lower part are engaged to a closed configuration. In some embodiments, the contact lens case is a sealable case for storing and transporting contact lenses in a contact lens solution. Various contact lens cases are described in, for example, USD Pat. No. 368368S; U.S. Pat. Nos. 5,131,532; 8,069,979; US Patent Publication 20120193245, incorporated herein by reference.

### 4.3. Topical Ophthalmic Cyclodextrin Solutions

In another aspect, the present disclosure further provides an ophthalmic cyclodextrin solution for use in treating a disorder or condition associated with wearing of contact lenses, as further provided in the detailed description herein.

In some embodiments, the ophthalmic cyclodextrin solution includes a disinfecting agent, antiseptic agent, and/or a preservative. In some embodiments, the ophthalmic cyclodextrin solution is substantially free of a disinfecting agent, antiseptic agent, and/or a preservative. In some embodiments, the ophthalmic cyclodextrin solution does not contain a disinfecting agent, antiseptic agent, and/or a preservative.

As discussed above, the term “substantially free of a disinfecting agent, antiseptic agent, and/or a preservative” refers to a concentration or amount of a disinfecting agent, antiseptic agent, and/or preservative that is ineffective in disinfection, antiseptic effects, and/or a preservative effects. In some embodiments, substantially free of a disinfecting agent, antiseptic agent, and/or a preservative refers to less than 0.005% w/v, less than 0.001% w/v, less than 0.0005% w/v, less than 0.0001% w/v, or less than 0.00005% w/v of a disinfecting agent, antiseptic agent and/or a preservative.

In some embodiments of the ophthalmic cyclodextrin solution, the cyclodextrin solution is substantially free of inclusion complexes, particularly substantially free of inclusion complexes formed with a pharmaceutically active agent. In some embodiments, the ophthalmic cyclodextrin solution is substantially free of a pharmaceutically active agent capable of forming an inclusion complex with the cyclodextrin. In some embodiments, the ophthalmic solution contains cyclodextrin as the sole or only pharmaceutically active agent in the ophthalmic solution.

In some embodiments, the ophthalmic cyclodextrin solution contains cyclodextrin in an amount effective to treat a disorder or condition associated with wearing a contact lens. In some embodiments, the concentration of cyclodextrin is sufficient to provide a mucomimetic layer or film on the eye surface or a contact lens when the contact lens is placed onto the eye.

In some embodiments, the concentration of cyclodextrin in the ophthalmic solution is effective to bind a biological mediator or effector of a disorder or condition associated with wearing a contact lens. In some the biological mediator is an allergen, prostaglandin, and/or a toxic aldehyde, for example malondialdehyde or 4-hydroxynonenal.

In some embodiments, the cyclodextrin for the ophthalmic solution is α-cyclodextrin, β-cyclodextrin, γ-cyclodextrin, derivatives thereof, or combinations thereof. In some embodiments, cyclodextrins derivatives useful for the ophthalmic solution include, among others, maltosyl, glucosyl, and maltotriosyl derivatives of β- and γ-cyclodextrins (see e.g., U.S. Pat. No. 5,024,998, incorporated herein by reference). Other useful cyclodextrins include cyclodextrins in which the hydroxyl groups on the glycosyl units have been modified, for example by amination, esterification or etherification.

In some embodiments, the ophthalmic solution comprises a cyclodextrin or derivative thereof selected from carboxyalkyl cyclodextrin, hydroxyalkyl cyclodextrin, sulfoalkylether cyclodextrin, and alkyl cyclodextrin. In various embodiments, the alkyl group in the cyclodextrin is methyl, ethyl, propyl, butyl, pentyl, or hexyl.

In some embodiments, the ophthalmic solution comprises α-cyclodextrin or a derivative thereof. In some embodiments, the α-cyclodextrin or a derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof. In some embodiments, the alkyl group in the α-cyclodextrin derivative is methyl, ethyl, propyl, butyl, pentyl or hexyl.

In some embodiments, the ophthalmic solution comprises β-cyclodextrin or a derivative thereof. In some embodiments, the β-cyclodextrin or derivative thereof is selected from carboxyalkyl-β-cyclodextrin, hydroxyalkyl-β-cyclodextrin, sulfoalkylether-β-cyclodextrin, alkyl-β-cyclodextrin, and combinations thereof. In some embodiments, the alkyl group in the β-cyclodextrin derivative is methyl, ethyl, propyl, butyl, pentyl or hexyl.

In some embodiments, the β-cyclodextrin or a derivative thereof is hydroxyalkyl-β-cyclodextrin or sulfoalkylether-β-cyclodextrin. In some embodiments, the hydroxyalkyl-β-cyclodextrin is hydroxypropyl-β-cyclodextrin. In some embodiments, the sulfoalkylether-β-cyclodextrin is sulfobutylether-β-cyclodextrin. In some embodiments, β-cyclodextrin or a derivative thereof is alkyl-β-cyclodextrin, in particular methyl-β-cyclodextrin. In some embodiments using methyl-β-cyclodextrin, the β-cyclodextrin is randomly methylated β-cyclodextrin.

In some embodiments, the ophthalmic solution comprises γ-cyclodextrin or a derivative thereof. In some embodiments, the γ-cyclodextrin or derivative thereof is selected from carboxyalkyl-γ-cyclodextrin, hydroxyalkyl-γ-cyclodextrin, sulfoalkylether-γ-cyclodextrin, and alkyl-γ-cyclodextrin. In some embodiments, the alkyl group in the γ-cyclodextrin derivative is methyl, ethyl, propyl, butyl, pentyl, or hexyl. In some embodiments, the γ-cyclodextrin or derivative thereof is hydroxyalkyl-γ-cyclodextrin or sulfoalkylether-γ-cyclodextrin. In some embodiments, the hydroxyalkyl-γ-cyclodextrin is hydroxypropyl-γ-cyclodextrin, such as 2-hydroxypropyl-γ-cyclodextrin.

In some embodiments, the ophthalmic solution comprises 6^{A},6^{B},6^{C},6^{D},6^{E},6^{F},6^{G},6^{H}-Octakis-S -(2-carboxyethyl)-6^{A},6^{B},6^{C},6^{D},6^{E},6^{F},6^{G},6^{H}-octathio-γ-cyclodextrin (i.e., S-2-carboxyethyl-octathio -γ-cyclodextrin, also referred to as sugammadex) and/or 6A,6^{B},6^{C},6^{D},6^{E},6^{F}, 6^{G}-Heptakis-S -(2-carboxyethyl)-6^{A},6^{B},6^{C},6^{D}, 6^{E}, 6^{F}6^{G}-heptathio-γ-cyclodextrin (i.e., S-2-carboxyethyl-heptathio-γ-cyclodextrin). Thus, in some embodiments, the cyclodextrin is 2-carboxyethyl-octathio-γ-cyclodextrin or 2-carboxyethyl-heptathio-γ-cyclodextrin.

In some embodiments, the cyclodextrin is a mixture of cyclodextrins. Such mixtures can be a combination of: α-cyclodextrin and β-cyclodextrin, including combinations of α-cyclodextrin and β-cyclodextrin derivatives; α-cyclodextrin and γ-cyclodextrin, including combinations of α-cyclodextrin and γ-cyclodextrin derivatives; β-cyclodextrin and γ-cyclodextrin, including combinations of β-cyclodextrin and γ-cyclodextrin derivatives; or α-cyclodextrin, β-cyclodextrin, and γ-cyclodextrin, including combinations of α-cyclodextrin, β-cyclodextrin, and γ-cyclodextrin derivatives.

In some embodiments, the ophthalmic solution contains a plurality of different cyclodextrins. In some embodiments, the ophthalmic solution contains a combination or mixture of different cyclodextrins. In some embodiments, the ophthalmic solution contains a first cyclodextrin and a second cyclodextrin, where the first cyclodextrin is different from the first cyclodextrin. In some embodiments, mixtures of cyclodextrins can be a combination of: α-cyclodextrin and β-cyclodextrin, including combinations of α-cyclodextrin and β-cyclodextrin derivatives; α-cyclodextrin and γ-cyclodextrin, including combinations of α-cyclodextrin and γ-cyclodextrin derivatives; β-cyclodextrin and γ-cyclodextrin, including combinations of β-cyclodextrin and γ-cyclodextrin derivatives; or α-cyclodextrin, β-cyclodextrin, and γ-cyclodextrin, including combinations of α-cyclodextrin, β-cyclodextrin, and γ-cyclodextrin derivatives.

In some embodiments, various salts of the cyclodextrin or salts of the cyclodextrin derivative can be used in the ophthalmic cyclodextrin solution. In some embodiments, the salts are pharmaceutically acceptable salt(s), which refers to those salts of compounds, i.e., cyclodextrin, that are safe and effective for use in mammals and that possess the desired biological activity. Pharmaceutically acceptable salts include salts of acidic or basic groups present in the cyclodextrins. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, and p-toluenesulfonate salts. Suitable base salts include, but are not limited to, aluminum, calcium, lithium, magnesium, potassium, sodium, zinc, and diethanolamine salts. In some embodiments, the cyclodextrin is in the form of a sodium or potassium salt. In some embodiments, the cyclodextrin is in the form an ophthalmically acceptable salt. Guidance on suitable pharmaceutically acceptable salts, ophthalmically acceptable salts, and their application to drug formulations can be found in various references, such as Remington's Pharmaceutical Sciences, 17th Ed., Mack Publishing Company, Easton, Pa., 1985, and Berge, et al., 1977, “Pharmaceutical Salts,” J Pharm Sci. 66:1-19, both of which are incorporated herein by reference.

In some embodiments, the ophthalmic solution contains cyclodextrin at about 0.1% w/v to about 50% w/v; about 0.5% w/v to about 45% w/v, about 1% w/v to about 40% w/v; about 2% w/v to about 35% w/v, about 5% w/v to about 30% w/v, about 10% w/v to about 25% w/v, or about 15% to about 20% w/v.

In some embodiments, the ophthalmic solution contains cyclodextrin at about 0.1% w/v, about 0.2% w/v, about 0.3%, w/v, about 0.4% w/v, about 0.5% w/v, about 0.6% w/v, about 0.7% w/v, about 0.8% w/v, about 0.9% w/v, about 1% w/v, about 1.5% w/v, about 2% w/v, about 3% w/v, about 4% w/v, about 5% w/v, about 6% w/v, about 7% w/v, about 8% w/v, about 9% w/v, about 10% w/v, about 15% w/v, about 20% w/v, about 25% w/v, about 30% w/v, about 35% w/v, about 40% w/v, about 45% w/v, or about 50% w/v.

In some embodiments, the ophthalmic solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

In some embodiments, the ophthalmic solution contains cyclodextrin at a concentration greater than 5% w/v, for example, about 6% w/v to about 50% w/v; about 7% w/v to about 50% w/v; about 8% w/v to about 50% w/v; about 9% w/v to about 50% w/v; about 10% w/v to about 50% w/v; about 15% w/v to about 45% w/v; about 20% w/v to about 40% w/v, or about 25% w/v to about 35% w/v.

In some embodiments, the ophthalmic solution contains cyclodextrin at about 15% w/v to about 50% w/v; about 20% w/v to about 50% w/v, or about 25% w/v to about 50% w/v, about 30% w/v to about 50% w/v, about 35% w/v to about 50% w/v, or about 40% w/v to about 50% w/v.

In some embodiments, the ophthalmic solution contains cyclodextrin at about 6% w/v to about 40% w/v; about 7% w/v to about 40% w/v; about 8% w/v to about 40% w/v; about 9% w/v to about 40% w/v; about 10% w/v to about 40% w/v; about 15% w/v to about 40% w/v; about 20% w/v to about 40% w/v, or about 25% w/v to about 40% w/v, about 30% w/v to about 40% w/v, about 35% w/v to about 40% w/v.

In some embodiments, the ophthalmic solution contains cyclodextrin at about 6% w/v, about 7% w/v, about 8% w/v, about 9% w/v, about 10% w/v, about 11% w/v, about 12% w/v, about 13% w/v, about 14% w/v, about 15% w/v, about 16% w/v, about 17% w/v, about 18% w/v, about 19% w/v, about 20% w/v, about 25% w/v, about 30% w/v, about 35% w/v, about 40% w/v, about 45% w/v, or about 50% w/v.

In some embodiments, the ophthalmic solution contains cyclodextrin at a concentration greater than 10% w/v, for example, about 11% w/v to about 50% w/v; about 12% w/v to about 50% w/v; about 13% w/v to about 50% w/v; about 14% w/v to about 50% w/v; about 15% w/v to about 50% w/v; about 16% w/v to about 50% w/v; about 17% w/v to about 50% w/v; about 18% w/v to about 50% w/v; about 19% w/v to about 50% w/v; about 20% w/v to about 50% w/v; about 25% w/v to about 45% w/v; or about 30% w/v to about 40% w/v.

In some embodiments, the ophthalmic solution contains cyclodextrin at a concentration greater than 10% w/v, for example, at about 11% w/v, about 12% w/v, about 13% w/v, about 14% w/v, about 15% w/v, about 16% w/v, about 17% w/v, about 18% w/v, about 19% w/v, about 20% w/v, about 25% w/v, about 30% w/v, about 35% w/v, about 40% w/v, about 45% w/v, or about 50% w/v.

In some embodiments, the cyclodextrin is present in the ophthalmic solution in the specified concentrations above. In some embodiments, the cyclodextrin is an α-cyclodextrin, β-cyclodextrin, γ-cyclodextrin, derivatives thereof, or combinations thereof at the specified concentrations above. In some embodiments, the β-cyclodextrin or derivative thereof is hydroxyalkyl-β-cyclodextrin or sulfoalkylether-β-cyclodextrin, particularly hydroxypropyl-β-cyclodextrin or sulfobutylether-β-cyclodextrin, at the specified concentrations above. In some embodiments, the cyclodextrin is a methyl-β-cyclodextrin, in particular randomly methylated-β-cyclodextrin, at the specified concentrations above. In some embodiments, the γ-cyclodextrin or derivative thereof is hydroxyalkyl-γ-cyclodextrin or sulfoalkylether-γ-cyclodextrin, particularly hydroxypropyl-γ-cyclodextrin or sulfobutylether-γ-cyclodextrin at the specified concentrations above. In some embodiments where the ophthalmic solution contains a mixture of cyclodextrins, the concentrations described above are for the total cyclodextrin in the ophthalmic solution.

In some embodiments, the ophthalmic solution contains a plurality of different cyclodextrins. In some embodiments, the ophthalmic cyclodextrin solution contains a combination or mixture of different cyclodextrins. In some embodiments, the ophthalmic cyclodextrin solution contains a first cyclodextrin and a second cyclodextrin, where the first cyclodextrin is different from the first cyclodextrin. In some embodiments, the first cyclodextrin is a γ-cyclodextrin, and the second cyclodextrin is a β-cyclodextrin. In some embodiments, the ophthalmic cyclodextrin solution contains a mixture of different β-cyclodextrins, for example a first cyclodextrin of sulfobutylether-β-cyclodextrin, and a second cyclodextrin of hydroxypropyl-β-cyclodextrin. In some embodiments where the ophthalmic cyclodextrin solution contains a mixture of cyclodextrins, the concentrations described above are for the total cyclodextrin in the contact lens solution.

In some embodiments, the ophthalmic solution contains in addition to the cyclodextrin, one or more ophthalmic pharmaceutically acceptable additive or excipient selected from a tonicity agent, buffering agent, wetting agent, viscosity enhancing agent, lubricating agent, chelating agent, antioxidant, pH adjusting agent, solubilizing agent, surfactant, chelating agent, emulsifying agent, suspending agent, and stabilizing agent. In some embodiments, the ophthalmic solution contains a disinfecting agent, antiseptic agent and/or a preservative, where appropriate.

In some embodiments, the ophthalmic solution can have one or more tonicity agents, which can be used to adjust the tonicity of the ophthalmic solution, for example to reduce irritation and make it compatible when the ophthalmic solution is administered to the eye, for example, to the tonicity of natural tears. Suitable tonicity agents include, by way of example and not limitation, dextrans (e.g., dextran 40 or 70), dextrose, glycerin, potassium chloride, propylene glycol, and sodium chloride. Equivalent amounts of one or more salts made up of cations, for example, such as potassium, ammonium and anions such as chloride, citrate, ascorbate, borate, phosphate, bicarbonate, sulfate, thiosulfate, bisulfate, the salts sodium bisulfate and ammonium sulfate, can also be used. The amount of tonicity agent will vary, depending on the particular agent to be added. In general, however, the compositions can have a tonicity agent in an amount sufficient to cause the final composition to have an ophthalmically acceptable osmolarity. In some embodiments, the ophthalmic cyclodextrin solution has an osmolarity of about 200 to about 1000 mOsm/L or about 200 to about 500 mOsm/L, or any specific value within said ranges (e.g., 200 mOsm/L, 210 mOsm/L, 220 mOsm/L, 230 mOsm/L, 240 mOsm/L, 250 mOsm/L, 260 mOsm/L, 270 mOsm/L, 280 mOsm/L, 290 mOsm/L, 300 mOsm/L, 310 mOsm/L, 320 mOsm/L, 330 mOsm/L, 340 mOsm/L, 350 mOsm/L, 360 mOsm/L, 370 mOsm/L, 380 mOsm/L, 390 mOsm/L or 400 mOsm/L). In a particular embodiment, the ophthalmic solutions are adjusted with a tonicity agent to an osmolarity ranging from about 250 to about 450 mOsm/L, or about 250 to about 350 mOsm/L.

In some embodiments, the ophthalmic solution contains one or more buffering agents for adjusting and/or maintaining the pH of the ophthalmic solution at a specified pH range. Generally, buffer capacity should be large enough to maintain the product pH for a reasonably long shelf-life but also low enough to allow rapid readjustment of the product to physiologic pH upon administration of the ophthalmic solution to the eye. Generally, buffer capacities of from about 0.01 to 0.1 can be used for ophthalmic solutions, particularly at concentrations that provide sufficient buffering capacity and minimizes adverse effects, e.g., irritation, to the eye. Exemplary buffering agents include, by way of example and not limitation, various salts (e.g., sodium, potassium, etc.), acids or bases, where appropriate, of the following agents, including, among others, acetate, borate, phosphate, bicarbonate, carbonate, citrate, tetraborate, biphosphate, tromethamine, hydroxyethyl morpholine, and THAM (trishydroxymethylamino-methane). In some embodiments, the buffering agent can be present from about 0.01 mM to about 100 mM, about 0.05 mM to about 100 mM, about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM. In some embodiments, the buffering agent can be present at about 0.01 mM, about 0.05 mM, about 0.1 mM, about 0.2 mM, about 0.5 mM, about 1 mM, about 5 mM, about 10 mM, about 20 mM, about 30 mM, about 40 mM, about 50 mM, or about 100 mM.

In some embodiments, an exemplary buffering agent is phosphate, particularly sodium phosphate, which can be prepared by standard procedures, for example by mixing appropriate amounts of one or more monobasic phosphates, dibasic phosphates, and the like. In particular, useful phosphate buffers are prepared from phosphate salts of alkali and/or alkaline earth metals, such as sodium or potassium phosphate, including sodium monobasic phosphate, sodium dibasic phosphate, potassium monobasic phosphate, and potassium dibasic phosphate. In some embodiments, the phosphate buffer can be present from about 0.5 mM to about 100 mM, from about 1 mM to about 50 mM, from about 1 mM to about 40 mM, from about 1 mM to about 30 mM, from about 1 mM to about 20 mM, or from about 1 mM to about 10 mM. In some embodiments, the phosphate buffer can be present at about 0.5 mM, about 1 mM, about 5 mM, about 10 mM, about 20 mM, about 30 mM, about 40 mM, about 50 mM, or about 100 mM.

In some embodiments, the ophthalmic solution contains one or more wetting agents. Generally, wetting agents generally are hydrophilic polymers, including, by way of example and not limitation, polysorbate 20 and 80, poloxamer 282, and tyloxapol. In some embodiments, wetting agents also include, among others, cellulose based polymers, such as hydroxypropylmethylcellulose (HPMC) and carboxymethylcellulose (CMC); polyvinylpyrrolidone; and polyvinyl alcohol. In some embodiments, the concentration of wetting agent, such as HPMC, ranges from about 0.1% to about 2% w/v, about 0.5% to about 1% w/v, or any specific value within the ranges. In some embodiments, the concentration of wetting agent, such as HPMC, ranges from about 0.1% to about 1.0% w/v, or any specific value within said range (e.g., 0.1-0.2%, 0.2-0.3%, 0.3-0.4%, 0.4-0.5%, 0.5-0.6%, 0.6-0.7%, 0.7-0.8%, 0.8-0.9%, 0.9-1.0%; about 0.2%, about 0.21%, about 0.22%, about 0.23%, about 0.24%, about 0.25%, about 0.26%, about 0.27%, about 0.28%, about 0.29%, about 0.30%, about 0.70%, about 0.71%, about 0.72%, about 0.73%, about 0.74%, about 0.75%, about 0.76%, about 0.77%, about 0.78%, about 0.79%, about 0.80%, about 0.81%, about 0.82%, about 0.83%, about 0.84%, about 0.85%, about 0.86%, about 0.87%, about 0.88%, about 0.89%, or about 0.90%).

In some embodiments, the ophthalmic solution contains one or more viscosity enhancing agents. The viscosity enhancing agent typically enhances the viscosity of the ophthalmic solution to increase retention time of the solution on the eye, and in some instances, to provide a protective layer on the eye surface. Viscosity enhancing agents include, among others, hyaluronate, hyaluronic acid or pharmaceutically acceptable salt thereof; cross-linked hyaluronic acid; carbopol gels, dextran (dextran 40, molecular weight of 40,000 Daltons; dextran 70, molecular weight of 70,000 Daltons), gelatin, glycerin, CMC, hydroxyethyl cellulose, HPMC, methylcellulose, ethylcellulose, polyethylene glycol, poloxamer 407, polysorbate 80, propylene glycol, polyvinyl alcohol, and polyvinylpyrrolidone (povidone), in various molecular weights and in various compatible combinations. In some embodiments, the ophthalmic compositions comprising the cyclodextrin has a viscosity that ranges from about 10 to about 150 centipoise (cP), about 15 to about 120 cP, about 20 to about 90 cP (or any specific value within said ranges). In some embodiments, the ophthalmic cyclodextrin solution has a viscosity that ranges from about 15 cP to about 30 cP, or any specific value within the range (i.e., about 15 cP, about 16 cP, about 17 cP, about 18 cP, about 19 cP, about 20 cP, about 20 cP, about 22 cP, about 23 cP, about 24 cP, about 25 cP, about 26 cP, about 27 cP, about 28 cP, about 29 cP, about 30 cP). In some embodiments, the ophthalmic solution comprising the cyclodextrin has a viscosity that ranges from about 70 cP to about 90 cP, or any specific value within said range (i.e., about 70 cP, about 71 cP, about 72 cP, about 73 cP, about 74 cP, about 75 cP, about 76 cP, about 77 cP, about 78 cP, about 79 cP, about 80 cP, about 81 cP, about 82 cP, about 83 cP, about 84 cP, about 85 cP, about 86 cP, about 87 cP, about 88 cP, about 89 cP or about 90 cP). In particular, a viscosity of from about 25 to about 50 cP are suitable for ophthalmic solutions.

In some embodiments, the amount of the viscosity enhancing agent is based on the agent used, and is in general in an amount of about 0.05 to 30% w/v. In some embodiments, the concentration of viscosity enhancing agent is about 0.05 to 30% w/v, about 0.1% w/v to about 25% w/v, about 0.25% w/v to about 15% w/v, about 0.5% w/v to about 15% w/v, about 0.75% w/v to about 10% w/v, or about 1.0% w/v to about 5% w/v.

In some embodiments, the amount of the viscosity enhancing agent in the ophthalmic solution is about 0.05% w/v to about 1.5% w/v; about 0.05% w/v to about 0.5% w/v; about 0.1% w/v to about 3.0% w/v; about 0.1% w/v to about 1.5% w/v; about 0.1% w/v to about 1.0% w/v; about 0.5% w/v to about 1% w/v; about 0.5% w/v to about 2.5% w/v; about 1.0% w/v to about 3.0% w/v; about 1.0% w/v to about 1.5% w/v; about 1.0% w/v to about 1.25% w/v; about 1.25% w/v to about 1.5% w/v; or about 1.5% w/v to about 3.0% w/v.

In some embodiments, the amount of the viscosity enhancing agent in the ophthalmic solution is about 0.1% w/v, about 0.25% w/v, about 0.5% w/v, about 0.75% w/v, about 1.0% w/v, about 1.1% w/v, about 1.15% w/v, about 1.20% w/v, about 1.25% w/v, about 1.30% w/v, about 1.35% w/v, about 1.40% w/v, about 1.45% w/v, about 1.5% w/v, about 2% w/v, about 3% w/v, about 4% w/v, about 5% w/v, about 10% w/v, about 15% w/v, about 20% w/v, about 25% w/v, or about 30% w/v.

In some embodiments, the molecular weight of a viscosity enhancing agent when polymeric is about 500,000 to about 5×10⁶ Daltons; about 500,000 Daltons to about 3×10⁶ Daltons; about 500,000 to about 2×10⁶ Daltons; about 500,000 to about 1×10⁶ Daltons; about 500,000 to about 2×10⁶ Daltons; about 1×10⁶ Daltons to about 3×10⁶ Daltons; about 1×10⁶ Daltons to about 2.5×10⁶ Daltons; about 1×10⁶ Daltons to about 2×10⁶ Daltons; or about 1.2×10⁶ Daltons to about 1.8×10⁶ Daltons. In some embodiments, the molecular weight is the number average molecular weight, and in other embodiments the molecular weight is the weight average molecular weight. Preferably the molecular weight is the number average molecular weight. In some embodiments, the viscosity enhancing agent comprises hyaluronate, hyaluronic acid or pharmaceutically acceptable salt thereof.

In some embodiments, the ophthalmic solution contains one or more ophthalmic lubricating agents. In some embodiments, the lubricants can approximate the consistency of endogenous tears and aid in natural tear build-up when the ophthalmic solution is administered to the eye. Lubricating agents can include non-phospholipid and phospholipid-based agents. In some embodiments, lubricants that are non-phospholipid based agents, including, but not limited to, propylene glycol; ethylene glycol; polyethylene glycol; hydroxypropylmethylcellulose; carboxymethylcellulose; hydroxypropylcellulose; dextrans, such as, dextran 70 and dextran 40; water soluble proteins, such as gelatin; vinyl polymers, such as polyvinyl alcohol, polyvinylpyrrolidone, povidone; petrolatum; mineral oil; and carbomers, such as, carbomer 934P, carbomer 941, carbomer 940, and carbomer 974P. Non-phospholipid lubricants can also include compatible mixtures of any of the foregoing agents.

In some embodiments, the ophthalmic solution contains one or more chelating agents. In some embodiments, the chelating agent is selected from ethylenediaminetetracetic acid (EDTA), ethylene glycol-bis(β-aminoethyl ether)-N,N,N′,N′-tetraacetic acid (EGTA), dihydroxy ethyl glycine, citric acid, or tartaric acid.

In some embodiments, the ophthalmic solution contains one or more disinfecting agent and/or antiseptic agent, where appropriate and compatible with contact lenses. In some embodiments, the disinfecting agent and/or antiseptic agent is selected from boric acid, hexamidine di-isetionate, polyaminopropyl-biguanide, polyhexamide, polyyquaternium, myristamidopropyl dimethylamine, and thimerosol.

In some embodiments, the ophthalmic solution can have one or more preservatives, for example, to extend shelf life or limit bacterial growth in the solutions during storage as well as when administered therapeutically onto the eye. Preservatives that can be used, include, among others, benzalkonium chloride, benzethonium chloride, benzododecinium bromide, cetylpyridinium chloride, chlorobutanol, thimerosol, phenylmercuric nitrate, phenylmercuric acetate, methyl/propylparabens, phenylethyl alcohol, sodium benzoate, sodium propionate, sorbic acid, and sodium perborate. The amount of preservative in the solution can be a level that enhances the shelf life, limits bacterial growth, or otherwise preserves the ophthalmic solution, with minimal toxicity to the eye tissues (see, e.g., The United States Pharmacopeia, 22nd rev., and The National Formulary, 17th Ed. Rockville, Md.). Concentration of preservative suitable for use in the ocular formulations can be determined by the person skilled in the art. In some embodiments, the preservatives can be used at an amount of from about 0.001% to about 1.0% w/v. For example, the preservative is present from about 0.005% to about 0.05% w/v, 0.005% to about 0.04% w/v, 0.01% to about 0.03% w/v, 0.01% to about 0.02% w/v, or from about 0.01% to about 0.015% w/v. In some embodiments, the amount of preservative can be about 0.005% w/v, about 0.01% w/v, about 0.012% w/v, about 0.014% w/v, about 0.016% w/v, about 0.018% w/v, about 0.02% w/v, about 0.03% w/v, about 0.04% w/v, or about 0.05% w/v. In some embodiments, no preservatives are used in the compositions.

In some embodiments, the ophthalmic solution can include one or more antioxidants. Suitable antioxidants, include, by way of example and not limitation, EDTA (e.g., disodium EDTA), sodium bisulphite, sodium metabisulphite, sodium thiosulfate, thiourea, and alpha-tocopherol.

In some embodiments, the ophthalmic solution can have a pH adjusting agent. In some embodiments, pH adjusting agents include, by way of example and not limitation, various salts (e.g., sodium, potassium, etc.), acids or bases, where appropriate, of the following agents, including, among others, acetate, borate, phosphate, bicarbonate, carbonate, citrate, tetraborate, biphosphate, tromethamine, hydroxyethyl morpholine, and THAM (trishydroxymethylamino-methane).

In some embodiments, the ophthalmic solution can have a solubilizing agent.

In some embodiments, the ophthalmic solution can have a surfactant. In some embodiments, exemplary surfactants include, among others, sorbitan esters such as and not limited to spans and mixtures of the partial esters of sorbitol and its mono- and di-anhydrides with oleic acid; polysorbates, such as and but not limited to, polysorbate 20 and 80; and poloxamers, such as but not limited to, poloxamer 282, pluronics and tyloxapol

In some embodiments, the ophthalmic solution can have an emulsifying agent. In some embodiments, the emulsifying agent can be oil in water where the oil phase is a medium chain triglyceride or one or more oils selected from the group consisting of olive, soy, corn, mineral, cottonseed, safflower, and sesame oil.

In some embodiments, the ophthalmic solution can have a stabilizing agent, suspending agent and viscosity modifying. This include but limited to among others, cellulose based polymers, such as hydroxyethylcellulose, hydroxypropylmethylcellulose (HPMC) and carboxymethylcellulose (CMC); polyvinylpyrrolidone; and polyvinyl alcohol.

In various embodiments, the pH of the ophthalmic cyclodextrin solution can be within 1.0 to 1.5 pH units from physiological pH, particularly the physiological pH in the external environment of the eye. The pH of human tears is approximately pH 7.4. Hence, the pH of the ophthalmic solution can be about 1.0 to 1.5 pH units above or below pH 7.4. In some embodiments, the pH of the ophthalmic solution is from about pH 6.0 to about pH 8.5. In some embodiments, the pH of the ophthalmic solution is from about pH 6.0 to about pH 8.0. In some embodiments, the pH of the ophthalmic solution is from about 6.5 to about 8.0. In some embodiments, the pH of the ophthalmic solution is from about 7.0 to about 8.0. In some embodiments, the pH of the ophthalmic solution is from about 7.0 to about 7.5. In some embodiments, the pH of the ophthalmic solution is about 6.5, about 7, about 7.5, about 8, or about 8.5. A person of skill in the art can select a pH that balances the stability of the ophthalmic solution and the tolerability of the eye to differences in pH from the natural condition. As is well known in the art, the pH of the solution can be adjusted by use of appropriate buffering agents and/or with an appropriate base (e.g., sodium hydroxide) or acid (e.g., hydrochloric acid).

In some embodiments, the ophthalmic solution comprises a cyclodextrin, and at least a viscosity enhancing agent, and a buffering agent. In some embodiments, the ophthalmic solution comprises a cyclodextrin, and at least a viscosity enhancing agent, a buffering agent, and optionally a disinfecting agent, antiseptic agent and/or a preservative. In some embodiments, the ophthalmic solution is substantially free of or does not contain a disinfecting agent, antiseptic agent and/or a preservative.

In some embodiments, the ophthalmic solution comprises a cyclodextrin, and at least a viscosity enhancing agent, a buffering agent, and a tonicity agent. In some embodiments, the ophthalmic solution comprises a cyclodextrin, and at least a viscosity enhancing agent, a buffering agent, a tonicity agent, and optionally a disinfecting agent, antiseptic agent and/or a preservative. In some embodiments, the contact lens solution is substantially free of or does not contain a disinfecting agent, antiseptic agent and/or a preservative.

In some embodiments, the ophthalmic solution comprises a cyclodextrin, and at least a wetting agent, and a buffering agent. In some embodiments, the ophthalmic solution comprises a cyclodextrin, and at least a wetting agent, a buffering agent, and optionally a disinfecting agent, antiseptic agent and/or a preservative. In some embodiments, the contact lens solution is substantially free of or does not contain a disinfecting agent, antiseptic agent and/or a preservative.

In some embodiments, the ophthalmic solution comprises a cyclodextrin, and at least a wetting agent, a buffering agent, and a tonicity agent. In some embodiments, the ophthalmic solution comprises a cyclodextrin, and at least a wetting agent, a buffering agent, a tonicity agent, and optionally a disinfecting agent, antiseptic agent and/or a preservative. In some embodiments, the ophthalmic solution is substantially free of or does not contain a disinfecting agent, antiseptic agent and/or a preservative.

In some embodiments, the ophthalmic solution comprises a cyclodextrin, and at least a viscosity enhancing agent and/or a wetting agent, and a buffering agent. In some embodiments, the ophthalmic solution comprises a cyclodextrin, and at least a viscosity enhancing agent and/or a wetting agent, a buffering agent, and optionally a disinfecting agent, antiseptic agent and/or a preservative. In some embodiments, the contact lens solution is substantially free of or does not contain a disinfecting agent, antiseptic agent and/or a preservative.

In some embodiments, the ophthalmic solution comprises a cyclodextrin, and at least a viscosity enhancing agent and/or a wetting agent, a buffering agent, and a tonicity agent. In some embodiments, the ophthalmic solution comprises a cyclodextrin, and at least a viscosity enhancing agent and/or a wetting agent, a buffering agent, a tonicity agent, and optionally a disinfecting agent, antiseptic agent and/or a preservative. In some embodiments, the contact lens solution is substantially free of or does not contain a disinfecting agent, antiseptic agent and/or a preservative.

In some embodiments, the ophthalmic solution comprises a cyclodextrin, and at least a lubricating agent, and a buffering agent. In some embodiments, the ophthalmic solution comprises a cyclodextrin, and at least a lubricating agent, a buffering agent, and optionally a disinfecting agent, antiseptic agent and/or a preservative. In some embodiments, the contact lens solution is substantially free of or does not contain a disinfecting agent, antiseptic agent and/or a preservative.

In some embodiments, the ophthalmic solution comprises a cyclodextrin, and at least a lubricating agent, a buffering agent, and a tonicity agent. In some embodiments, the ophthalmic solution comprises a cyclodextrin, and at least a lubricating agent, a buffering agent, a tonicity agent, and optionally a disinfecting agent, antiseptic agent and/or a preservative. In some embodiments, the contact lens solution is substantially free of or does not contain a disinfecting agent, antiseptic agent and/or a preservative.

In some embodiments where the ophthalmic solution is substantially free of a disinfecting agent, antiseptic agent and/or a preservative, the ophthalmic solution is in a single use vial or bottle. In particular, the ophthalmic solution is in a single use disposable squeeze vial, for example, with a snap off cap.

### 4.4. Treatment of Disorders or Conditions Associated With Contact Lenses

In some embodiments, the ophthalmic solution comprising a cyclodextrin is used to treat a disorder or condition associated with wearing a contact lens. In addition to its mucomimetic properties, cyclodextrin can bind certain mediators of the inflammatory response, such as prostaglandins, reactive aldehydes, and lipid peroxidation products (see e.g., Ao et al., 2016, Biol. Pharm. Bull, 39:1029-1034; Nociari et al., 2014, Proc Natl Acad Sci. USA, E1402-E1408; Pizzimenti et al., 2015, J Biomed Nanotechnol., 11(12):2169-85). Cyclodextrins can also bind cholesterol, acting as a sink to reduce biological activity associated with cholesterol levels (see Gasper et al., 2017, Scientific Reports, 7:2197; Irie et al., 1992, J. Pharm. Sci. 81(6):521e523). Clinically, cyclodextrin alone, in the absence of any pharmaceutically active agent, can, when administered topically, reduce the severity of allergic conjunctivitis. While this effect can be attributable to sequestration by cyclodextrin of allergens, topical cyclodextrin can also reduce amyloid-β and inflammation in eyes of mouse model of macular degeneration (see Kam et al., 2015, Experimental Eye Research 135:59e66; Patent Publication WO2017147617). Cyclodextrin's effect on severity of allergic conjunctivitis and eye inflammation in mouse indicate bioavailability of topical cyclodextrin on the eye. In addition, membrane permeability can be compromised by wearing a contact lens, and particularly when the eye is affected by disorders and conditions associated with wearing a contact lens, which can provide additional pathways for the cyclodextrin to bind biological effectors involved in the disorders or conditions.

In some embodiments, the ophthalmic solution comprising a cyclodextrin is used for treating a disorder or condition associated with wearing a contact lens. In some embodiments, a method of treating a disorder or condition associated with wearing a contact lens comprises topically administering to the eye of a patient in need thereof a therapeutically effective amount of an ophthalmic solution comprising a cyclodextrin. In some embodiments, any of the ophthalmic cyclodextrin formulations described herein can be used.

In some embodiments, the ophthalmic solution comprising a cyclodextrin is used to prevent occurrence, including recurrence, or reduce the risk of a disorder or condition associated with wearing a contact lens. In some embodiments, a method of preventing occurrence, including recurrence, or reducing the risk of a disorder or condition associated with wearing a contact lens comprises administering a therapeutically effective amount of an ophthalmic cyclodextrin solution as described herein.

In some embodiments, a therapeutically effective amount or an effective amount refers to an amount of ophthalmic cyclodextrin solution which is sufficient to eliminate or reduce a symptom of the specified disorder or condition. In some embodiments, the therapeutically effective amount is the amount sufficient for the treatment or prevention of the specified indication. “Treatment” in this context refers to reducing, ameliorating or mitigating one or more symptom of disorder or condition, as described in the present disclosure.

In some embodiments, “prevention” or “prophylactic treatment” refers to a reduction in the frequency of, or a delay in the onset of, symptoms associated with a disorder or condition associated with wearing a contact lens relative to a patient who does not receive the composition. In some embodiments, the ophthalmic cyclodextrin solution can be administered prophylactically to a patient previously diagnosed but showing no symptoms of the disorder or condition. The prophylactic treatment can follow the dosages and administration schedules used for treating a subject with the disorder or condition.

In some embodiments, the ophthalmic cyclodextrin solution is administered to the eye of the patient in need thereof. In some embodiments, the ophthalmic cyclodextrin solution is administered to the eye of the patient with the contact lens on the eye. In some embodiments, the ophthalmic cyclodextrin solution is administered to the eye without the contact lens on the eye.

In some embodiments, the contact-lens associated disorder or condition for prevention or treatment is, among others, corneal edema, superficial keratitis, non-microbial associated red eye (contact lens acute red eye), endothelial polymegethism, giant papillary conjunctivitis, corneal neovascularization, meibomitis, allergy aggravation, pingueculitis, ptosis, contact lens-induced peripheral ulcer, deep stromal corneal opacities, corneal hypoxia, epithelial microcysts, excess mucus production, or eye dryness resulting from wearing of contact lens.

In some embodiments, the contact lens associated disorder or condition is corneal edema related to wearing a contact lens.

In some embodiments, the contact lens associated disorder or condition is superficial keratitis related to wearing a contact lens.

In some embodiments, the contact les associated disorder or condition is non-microbial associated red eye related to wearing a contact lens, also referred to as contact lens acute eye.

In some embodiments, the contact lens associated disorder or condition is endothelial polymegethism related to wearing a contact lens.

In some embodiments, the contact lens associated disorder or condition is giant papillary conjunctivitis related to wearing a contact lens.

In some embodiments, the contact lens associated disorder or condition is corneal neovascularization related to wearing a contact lens.

In some embodiments, the contact lens associated disorder or condition is meibomitis related to wearing a contact lens.

In some embodiments, the contact lens associated disorder or condition is allergy aggravation related to wearing a contact lens.

In some embodiments, the contact lens associated disorder or condition is pingueculitis related to wearing a contact lens.

In some embodiments, the contact lens associated disorder or condition is ptosis related to wearing a contact lens.

In some embodiments, the contact lens associated disorder or condition is contact lens-induced peripheral ulcer.

In some embodiments, the contact lens associated disorder or condition is deep stromal corneal opacities related to wearing a contact lens.

In some embodiments, the contact lens associated disorder or condition is corneal hypoxia related to wearing a contact lens.

In some embodiments, the contact lens associated disorder or condition is epithelial microcysts associated with wearing a contact lens.

In some embodiments, the contact lens associated disorder or condition is excess mucus production related to wearing a contact lens.

In some embodiments, the contact lens associated disorder or condition is dry eye syndrome related to wearing a contact lens.

In some embodiments, the dosages of the ophthalmic cyclodextrin solution can be selected based on a number of factors including, among others, the disorder or condition, the severity of the disorder or condition, age, sex, species, and condition of the patient. Effective amounts can also be extrapolated from dose-response curves derived from in vitro test systems, from animal models or clinical trials. It is to be further understood that for any particular patient, specific dosage regimens can be adjusted over time according to the subject's need and the professional judgment of the person administering or supervising the administration of the ophthalmic cyclodextrin solution.

In some embodiments, the ophthalmic cyclodextrin solution is administered as needed, for example based on guidance from a medical professional skilled in the art, and/or as assessed by the patient. In some embodiments, the ophthalmic cyclodextrin solution is administered at least once per week. In some embodiments, the ophthalmic cyclodextrin solution is administered at least once every two days. In some embodiments, the ophthalmic cyclodextrin solution is administered at least once per day. In some embodiments, the ophthalmic cyclodextrin solution is administered at least twice per day, at least three times per day, at least four times per day, at least five times per day, or at least six times per day.

For topical ophthalmic administration, each administration comprises one or more aliquots of the ophthalmic cyclodextrin solution. Each aliquot can be a defined volume, for example about 10 μL to about 100 μL, about 20 μL to about 80 μL, or about 30 μL to about 60 μL. In some embodiments, each aliquot is about 10 μL, about 20 μL, about 30 μL, about 40 μL, about 50 μL, about 60 μL, about 70 μL, about 80 μL, about 90 μL, or about 100 μL. In some embodiments, the aliquot administered in an estimated volume, for example an applied drop using a dropper or a squeeze vial. In some embodiments, one or more drops, at least two drops, at least three drops, at least 4 drops, at least 5 drops, or at least 6 drops are topically applied to an eye or to each of both eyes at each administration. In some embodiments, each administration comprises sequential administration, for example, a first administration of one or more aliquots (e.g., one or more drops), a first time period for allowing absorption of the composition, followed by a second administration of one or more aliquots (e.g., one or more drops).

Generally, in various embodiments, the ophthalmic cyclodextrin solution is allowed to remain in contact with the eye for a therapeutically effective time period. In some embodiments, the composition is allowed to remain in contact with the eye for at least about 0.1 min, 0.2 min 0.25 min, 0.3 min, 0.5 min, 1 min, 1.5 min, 2 min or longer, 5 min or longer, 10 min or longer for example, as determined by the medical professional skilled in the art.

In some embodiments, the treatment duration is for a time resulting in reducing, ameliorating, or mitigating one or more symptoms of a disorder or condition associated with wearing of a contact lens. In some embodiments, the symptoms to be assessed include one or more of: inflammation in the eye, the amount of tears, level of itchiness, improvements in vision, and swelling of the eyelid. In some embodiments, the duration of treatment is at least 2 days, at least 3 days, at least 5 days, at least 7 days (i.e., one week), at least 10 days, at least 14 days (i.e., two weeks), at least 17 days, at least 21 days (i.e., three weeks), or at least 28 days (i.e., four weeks) or more. In some embodiments, the treatment duration is one month or more, two months or more, three months or more, or four months or more. In some embodiments, the ophthalmic solution is administered as long as needed.

In some embodiments, the duration of treatment is about 2 days to about 4 months, about 7 days (i.e., one week) to about 3 months, about 14 days (i.e., two weeks) to about 2 months, or about 21 days (i.e., three weeks) to about 6 weeks (i.e., 1.5 months).

### 4.5. Combination Products, Contact Lens Packages or Contact Lens Kits

In another aspect, the cyclodextrin contact lens solution and the ophthalmic cyclodextrin solution described herein can be provided as a combination, e.g., combination product, combination package or a kit.

In some embodiments, the combination, for example a combination product, package or kit, comprises: (a) a first contact lens solution comprising cyclodextrin and at least a disinfecting agent, antiseptic agent, and/or a preservative; and (b) a second contact lens solution comprising cyclodextrin, wherein the second contact lens solution is substantially free of or does not contain a disinfecting agent, antiseptic agent, and/or a preservative, as discussed above.

In some embodiments, the combination, for example a combination product, package or kit, comprises: (a) a first contact lens solution comprising cyclodextrin, wherein the first contact lens solution is substantially free of or does not contain a disinfecting agent, antiseptic agent, and/or a preservative; and (b) a second contact lens solution comprising cyclodextrin, wherein the second contact lens solution is substantially free of or does not contain a disinfecting agent, antiseptic agent, and/or a preservative, as discussed above.

In some embodiments, the combination, for example a combination product, package or kit, comprises: (a) a first contact lens solution comprising a cyclodextrin and at least a disinfecting agent, antiseptic agent and/or a preservative; (b) a contact lens, wherein the contact lens is in the first contact lens solution; (c) a second contact lens solution comprising cyclodextrin, wherein the second contact lens solution is substantially free of or does not contain a disinfecting agent, antiseptic agent and/or a preservative.

In some embodiments, the combination, for example a combination product, package or kit, comprises: (a) a first contact lens solution comprising cyclodextrin and at least a disinfecting agent, antiseptic agent and/or a preservative; and (b) an ophthalmic solution comprising a cyclodextrin, wherein the ophthalmic solution has cyclodextrin as the only or sole pharmaceutically active agent. In some embodiments, the ophthalmic solution is substantially free of or does not contain a disinfecting agent, antiseptic agent, and/or a preservative.

In some embodiments, the combination, for example a combination product, package or kit, comprises: (a) a first contact lens solution comprising a cyclodextrin and at least a disinfecting agent, antiseptic agent and/or a preservative; (b) a contact lens, wherein the contact lens is in the first contact lens solution; (c) an ophthalmic solution comprising a cyclodextrin. In some embodiments, the ophthalmic solution is substantially free of or does not contain a disinfecting agent, antiseptic agent and/or a preservative.

In some embodiments of the combination containing a contact lens, the contact lens is a soft lens, for example a hydrogel contact lens or a silicon hydrogel lens; gas permeable contact lens; hybrid contact lens (e.g., contact lens comprised of a rigid gas permeable central zone, surrounded by a “skirt” of hydrogel or silicone hydrogel material); or rigid lenses, such as polymethyl methacrylate (PMMA) contact lens. In some embodiments, the contact lenses can be disposable or reusable.

In some embodiments of the combination containing a contact lens, the combination further comprises a contact lens case, wherein the contact lens case has one or more compartments or chambers for storing the contact lens in a contact lens storage solution, such as the cyclodextrin contact lens solutions described herein. In some embodiments, the contact lens case comprises a first compartment or chamber for storage of a contact lens, and a second compartment or chamber which can receive a contact lens and a contact lens solution. In some embodiments, the contact lens case comprises an upper part and a lower part, wherein the upper part is connected to the lower part by a hinge, and the lower part is configured to have one or more compartments or chambers capable of receiving a contact lens, wherein various solutions can be placed into the compartment or chamber, and the upper part moves relative to the lower part and forms a lid when the upper part and the lower part are engaged to a closed configuration. In some embodiments, the contact lens case is a sealable case for storing and transporting contact lenses in a contact lens solution.

In some embodiments, the second contact lens solution, particularly when packaged together with the first contact lens solution is contained in a single use container, or a multi-use container. In some embodiments, the single use container is sterile and all of the composition in the package is intended to be consumed in a single application, for example for rinsing the contact lens prior to applying the contact lens to the surface of the eye. In some embodiments, the single use contact lens solution is contained in a single use, disposable squeeze vial, in particular with a non-resealable snap cap or tear off cap. In some embodiments, the combination product, package or kit comprises a plurality of single use, disposable squeeze vials of the contact lens solution.

In some embodiments, the ophthalmic cyclodextrin solution is contained in a single use container, or a multi-use container. In some embodiments, the single use container is sterile and is intended to be used in a single application. In some embodiments, the single use ophthalmic cyclodextrin solution is contained in a single use, disposable squeeze vial, in particular a non-resealable snap cap or tear off cap. In some embodiments, the combination product, package or kit comprises a plurality of single use, disposable squeeze vials of the ophthalmic cyclodextrin solution.

Packaging of the cyclodextrin compositions as single dose product can reduce or eliminate the need for a disinfectant, antiseptic, and/or preservative in the composition, where if present may cause ocular irritation, particularly in patients suffering from pre-existing ocular irritation.

While the contact lens solution and the ophthalmic solution are preferably formulated as ready to use aqueous solutions i.e., solution which does not require any dilution or preparation before use, alternative formulations can be used. For example, the contact lens solution and/or ophthalmic solution can be provided in a lyophilized or as a dried powder or solid form ready for reconstitution with a solvent, such as sterile water (e.g., deionized or distilled) or buffer solution. In some embodiments, the contact lens solution and the ophthalmic cyclodextrin solution are pyrogen and/or endotoxin free. In various embodiments, the sterile cyclodextrin compositions can be prepared by appropriate sterilization procedures known in the art. In some embodiments, the cyclodextrin or derivative thereof is produced under sterile conditions, and the mixing and packaging is conducted under sterile conditions. In some embodiments, the compositions of may be filter-sterilized and filled in vials, including unit dose vials providing sterile unit dose formulations. In some embodiments, the composition and/or agents of the compositions is sterilized by steam, γ-radiation, or by appropriate chemical sterilization procedures.

In some embodiments, the combination, for example as a combination product, package or kit further comprises instructions for proper use of the contact lens solution, contact lenses (if present), and/or ophthalmic cyclodextrin solution, as well as description of side effects and dosage information and administration. While the instructional materials typically comprise written or printed materials, any medium capable of storing such instructions and communicating them to an end user is contemplated. Such media include, but are not limited to, electronic storage media (e.g., magnetic discs, tapes, cartridges, chips), optical media (e.g., CD ROM), and the like. Such media may include addresses to internet sites that provide such instructional materials, including downloadable instructions.

All publications, patents, patent applications and other documents cited in this application are hereby incorporated by reference in their entireties for all purposes to the same extent as if each individual publication, patent, patent application or other document were individually indicated to be incorporated by reference for all purposes.

What is claimed is:

1. A contact lens kit comprising:
(a) one or more contact lenses stored in an contact lens solution comprising a cyclodextrin; and
(b) a topical ophthalmic solution comprising a cyclodextrin as the only active agent and optionally one or more ophthalmically acceptable excipients.

2. The kit of claim 1, wherein the cyclodextrin in the topical ophthalmic solution is capable of complexing an allergen.

3. The kit of claim 1, wherein the cyclodextrin or derivative thereof in the topical ophthalmic composition is selected from α-cyclodextrin, β-cyclodextrin, γ-cyclodextrin, derivatives thereof, and combinations thereof.

4. The kit of claim 3, wherein the cyclodextrin or derivative thereof in the topical ophthalmic composition is selected from carboxyalkyl cyclodextrin, hydroxyalkyl cyclodextrin, sulfoalkylether cyclodextrin, alkyl cyclodextrin, and combinations thereof.

5. The kit of claim 4, wherein the cyclodextrin or derivative thereof in the topical ophthalmic solution comprises β-cyclodextrin or a derivative thereof.

6. The kit of claim 4, wherein the β-cyclodextrin or derivative thereof in the topical ophthalmic solution is selected from carboxyalkyl-β-cyclodextrin, hydroxyalkyl-β-cyclodextrin, sulfoalkylether-β-cyclodextrin, alkyl-β-cyclodextrin, and combinations thereof.

7. The kit of claim 6, wherein the β-cyclodextrin or derivative thereof in the topical ophthalmic solution is sulfoalkylether-β-cyclodextrin, hydroxyalkyl-β-cyclodextrin, or combinations thereof.

8. The kit of claim 7, wherein the sulfoalkylether-β-cyclodextrin in the topical ophthalmic solution is sulfobutylether-β-cyclodextrin.

9. The kit of claim 8, wherein the hydroxyalkyl-β-cyclodextrin in the topical ophthalmic solution is hydroxypropyl-β-cyclodextrin.

10. The kit of any one of claims 1 to 9, wherein the topical ophthalmic solution contains cyclodextrin at a concentration of about 0.1 w/v to about 50% w/v, about 0.1 w/v to about 20% w/v, about 0.5% w/v to about 10% w/v, or about 1% to about 5% w/v.

11. The kit of any one of claims 1 to 9, wherein the topical ophthalmic solution contains cyclodextrin at a concentration of about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, about 30% w/v, about 40%, or about 50%.

12. The kit of any one of claims 1 to 9, wherein the topical ophthalmic solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

13. The kit of any one of claims 1 to 12, wherein the topical ophthalmic solution further comprises a pharmaceutically acceptable excipient selected from a viscosity enhancing agent, tonicity agent, preservative, buffering agent, pH adjusting agent, solubilizing agent, surfactant, chelating agent, emulsifying agent, suspending agent, stabilizing agent, and antioxidant.

14. The kit of claim 13, wherein the ophthalmic pharmaceutically acceptable excipient comprises a buffering agent.

15. The kit of any one of claims 1 to 14, wherein the topical ophthalmic solution has a pH of about 6.5 to about 8.5.

16. The kit of any one of claims 1 to 15, wherein the topical ophthalmic solution is in a single-use vial.

17. The kit of claim 16, wherein the single use vial comprises a disposable plastic squeeze vial with a non-resealable snap-off or tear-off cap.

18. The kit of any one of claims 1 to 17, wherein the cyclodextrin in the contact lens solution is selected from α-cyclodextrin, β-cyclodextrin, γ-cyclodextrin, derivatives thereof, and combinations thereof.

19. The kit of claim 18, wherein the cyclodextrin in the contact lens solution is selected from carboxyalkyl cyclodextrin, hydroxyalkyl cyclodextrin, sulfoalkylether cyclodextrin, alkyl cyclodextrin, and combinations thereof.

20. The kit of claim 19, wherein the cyclodextrin in the contact lens solution comprises β-cyclodextrin or a derivative thereof.

21. The kit of claim 20, wherein the β-cyclodextrin in the contact lens solution is selected from carboxyalkyl-β-cyclodextrin, hydroxyalkyl-β-cyclodextrin, sulfoalkylether-β-cyclodextrin, alkyl-β-cyclodextrin, and combinations thereof.

22. The kit of claim 20, wherein the β-cyclodextrin in the contact lens solution is sulfoalkylether-β-cyclodextrin or hydroxyalkyl-β-cyclodextrin.

23. The kit of claim 22, wherein the sulfoalkylether-β-cyclodextrin is sulfobutylether-β-cyclodextrin.

24. The kit of claim 22, wherein the hydroxyalkyl-β-cyclodextrin is hydroxypropyl-β-cyclodextrin.

25. The kit of any one of claims 1 to 24, wherein the contact lens solution contains cyclodextrin at a concentration of about 0.1 to about 30% w/v, about 0.1 to about 20% w/v, 0.5% to about 10% w/v, or about 1% to about 5% w/v.

26. The kit of any one of claims 1 to 24, wherein the contact lens solution contains cyclodextrin at a concentration of about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, about 30% w/v, about 40% w/v, or about 50% w/v.

27. The kit of any one of claims 1 to 24, wherein the contact lens solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

28. The kit of any one of claims 1 to 27, wherein the contact lens solution further comprises one or more ophthalmically acceptable excipients.

29. The kit of claim 28, wherein the ophthalmically acceptable excipient in the contact lens solution is selected from a viscosity enhancing agent, tonicity agent, preservative, buffering agent, pH adjusting agent, solubilizing agent, surfactant, chelating agent, emulsifying agent, suspending agent, stabilizing agent, and antioxidant.

30. The kit of claim 29, wherein the ophthalmically acceptable excipient in the contact lens solution is a viscosity enhancing agent or a suspending agent.

31. The kit of claim 30, wherein the viscosity enhancing agent or suspending agent is selected from polyvinylpyrrolidone (PVP), polyvinyl alcohol (PVA), hydroxypropyl methylcellulose (HPMC), hydroxy propyl cellulose (HPC), carbomer, dextran, and combinations thereof.

32. The kit of any one of claims 28 to 31, wherein the ophthalmically acceptable excipient comprises a buffering agent.

33. The kit of claim 32, wherein the buffering agent is phosphate.

34. The kit of claim 33, wherein the phosphate is sodium phosphate.

35. The kit of any one of claims 1 to 34, wherein the contact lens solution has a pH of about 6.5 to about 8.5.

36. The kit of any one of claims 1 to 35, wherein the contact lens solution is substantially free of a disinfecting agent, antiseptic agent and/or a preservative.

37. The kit of any one of claims 1 to 35, wherein the contact lens solution further includes a disinfecting agent, antiseptic agent and/or a preservative.

38. A contact lens solution comprising cyclodextrin, wherein the solution is substantially free of a disinfecting agent, antiseptic agent and/or a preservative.

39. The contact lens solution of claim 38, wherein the cyclodextrin in the contact lens solution is substantially free of inclusion complexes.

40. The contact lens solution of claim 38 or 39, wherein the cyclodextrin is selected from α-cyclodextrin, β-cyclodextrin, γ-cyclodextrin, derivatives thereof, and combinations thereof.

41. The contact lens solution of claim 40, wherein the cyclodextrin is selected from carboxyalkyl cyclodextrin, hydroxyalkyl cyclodextrin, sulfoalkylether cyclodextrin, alkyl cyclodextrin, and combinations thereof.

42. The contact lens solution of claim 40, wherein the cyclodextrin comprises β-cyclodextrin or a derivative thereof.

43. The contact lens solution of claim 42, wherein the β-cyclodextrin is selected from carboxyalkyl-β-cyclodextrin, hydroxyalkyl-β-cyclodextrin, sulfoalkylether-β-cyclodextrin, cyclodextrin, and combinations thereof.

44. The contact lens solution of claim 43, wherein the β-cyclodextrin is sulfoalkylether-β-cyclodextrin or hydroxyalkyl-β-cyclodextrin.

45. The contact lens solution of claim 44, wherein the sulfoalkylether-β-cyclodextrin is sulfobutylether-β-cyclodextrin.

46. The contact lens solution of claim 44, wherein the hydroxyalkyl-β-cyclodextrin is hydroxypropyl-β-cyclodextrin.

47. The contact lens solution of any one of claims 38 to 46, wherein the cyclodextrin is at a concentration of about 0.1 w/v to about 50% w/v, about 0.1 w/v to about 20% w/v, about 0.5% w/v to about 10% w/v, or about 1% w/v to about 5% w/v.

48. The contact lens solution of any one of claims 38 to 46, wherein the cyclodextrin is at a concentration of about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, about 30% w/v, about 40% w/v, or about 50% w/v.

49. The contact lens solution of any one of claims 38 to 46, wherein the contact lens solution contains cyclodextrin at a concentration greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

50. The contact lens solution of any one of claims 38 to 49, further comprising one or more ophthalmically acceptable excipients.

51. The contact lens solution of claim 50, wherein the ophthalmically acceptable excipient is selected from a viscosity enhancing agent, tonicity agent, buffering agent, pH adjusting agent, solubilizing agent, surfactant, emulsifying agent, suspending agent, and stabilizing agent.

52. The contact lens solution of claim 51, wherein the ophthalmically acceptable excipient comprises a viscosity enhancing agent or suspending agent.

53. The contact lens solution of claim 52, wherein the viscosity enhancing agent or suspending agent is selected from polyvinylpyrrolidone (PVP), polyvinyl alcohol (PVA), hydroxypropyl methylcellulose (HPMC), hydroxy propyl cellulose (HPC), carbomer, dextran, and combinations thereof.

54. The contact lens solution of any one of claims 51 to 53, wherein the ophthalmically acceptable excipient comprises a buffering agent.

55. The contact lens solution of claim 54, wherein the buffering agent is phosphate.

56. The contact lens solution of claim 55, wherein the phosphate is sodium phosphate.

57. The contact lens solution of any one of claims 38 to 56, wherein the solution has a pH of about 6.5 to about 8.5.

58. A method of preventing, reducing the risk of, or treating contact-lens associated inflammatory condition in a patient wearing contact lens, comprising topically administering to the eye of a patient in need thereof a therapeutically effective amount of an ophthalmic solution comprising a cyclodextrin as the sole or only pharmaceutically active agent.

59. The method of claim 58, wherein the cyclodextrin is substantially free of inclusion complexes.

60. The method of any one of claims 58 to 59, wherein the cyclodextrin comprises α-cyclodextrin, β-cyclodextrin, γ-cyclodextrin, derivatives thereof, and combinations thereof.

61. The method of claim 60, wherein the cyclodextrin is selected from carboxyalkyl cyclodextrin, hydroxyalkyl cyclodextrin, sulfoalkylether cyclodextrin, alkyl cyclodextrin, and combinations thereof.

62. The method of claim 60, wherein the cyclodextrin comprises β-cyclodextrin, or a derivative thereof.

63. The method of claim 62, wherein the β-cyclodextrin is selected from carboxyalkyl-β-cyclodextrin, hydroxyalkyl-β-cyclodextrin, sulfoalkylether-β-cyclodextrin, alkyl-β-cyclodextrin, and combinations thereof.

64. The method of claim 62, wherein the β-cyclodextrin is sulfoalkylether-β-cyclodextrin, hydroxyalkyl-β-cyclodextrin, or combinations thereof.

65. The method of claim 64, wherein the sulfoalkylether-β-cyclodextrin is sulfobutylether-β-cyclodextrin.

66. The method of claim 64, wherein the hydroxyalkyl-β-cyclodextrin is hydroxypropyl-β-cyclodextrin.

67. The method of any one of claims 58 to 66, wherein the cyclodextrin is present at about 0.1 to about 50% w/v, about 0.1 to about 20% w/v, 0.5% to about 10% w/v, or about 1% to about 5% w/v.

68. The method of any one of claims 58 to 66, wherein the cyclodextrin is present at about 0.1% w/v, about 0.2% w/v, about 0.5% w/v, about 1% w/v, about 2% w/v, about 3% w/v, about 4% w/v, about 5% w/v, about 6% w/v, about 7% w/v, about 8% w/v, about 9% w/v, about 10% w/v, about 12% w/v, about 14% w/v, about 16% w/v, about 18% w/v, about 20% w/v, about 25% w/v, about 30% w/v, about 40% w/v, or about 50% w/v.

69. The method of any one of claims 58 to 66, wherein the cyclodextrin is present at greater than 5% w/v, greater than 6% w/v, greater than 7% w/v, greater than 8% w/v, greater than 9% w/v, greater than 10% w/v, greater than 11% w/v, greater than 12% w/v, greater than 13% w/v, greater than 14% w/v, greater than 15% w/v, greater than 16% w/v, greater than 17% w/v, greater than 18% w/v, greater than 19% w/v, greater than 20% w/v, greater than 25% w/v, greater than 35% w/v, or greater than 40% w/v, up to about 50% w/v.

70. The method of any one of claims 58 to 69, wherein the ophthalmic solution further comprises one or more ophthalmically acceptable excipients.

71. The method of claim 70, wherein the ophthalmically acceptable excipient is selected from a viscosity enhancing agent, tonicity agent, preservative, buffering agent, pH adjusting agent, solubilizing agent, surfactant, chelating agent, emulsifying agent, suspending agent, stabilizing agent, and antioxidant.

72. The method of claim 71, wherein the ophthalmically acceptable excipient comprises a viscosity enhancing agent or suspending agent.

73. The method of claim 72, wherein the viscosity enhancing agent or suspending agent is selected from polyvinylpyrrolidone (PVP), polyvinyl alcohol (PVA), hydroxypropyl methylcellulose (HPMC), hydroxy propyl cellulose (HPC), carbomer, dextran, and combinations thereof.

74. The method of claim 71, wherein the ophthalmically acceptable excipient comprises a buffering agent.

75. The method of any one of claims 58 to 68, wherein the topical ophthalmic solution has a pH of about 6.5 to about 8.5.

76. The method of any one of claims 58 to 75, wherein the topical ophthalmic solution is contained in a single-use vial.

77. The method of claim 76, wherein the single use vial comprises a disposable plastic squeeze vial with a non-resealable snap-off or tear-off cap.

78. The method of any one of claims 58 to 77, wherein contact-lens associated disorder or condition is corneal edema, superficial keratitis, non-microbial associated red eye (contact lens acute red eye), endothelial polymegethism, giant papillary Conjunctivitis, corneal neovascularization, meibomitis, allergy aggravation, pingueculitis, ptosis, contact lens-induced peripheral ulcer, deep stromal corneal opacities, corneal hypoxia, epithelial microcysts, excess mucus production, or eye dryness.
